(19) United States
(12) Patent Application Publication
(10) Pub. No.: US 2024/0017254 A1
(43) Pub. Date: Jan. 18, 2024

(54) SYSTEM FOR DETECTION OF A TARGET IN A LIQUID SAMPLE

(71) Applicant: LUMIRADX UK LTD, London (GB)

(72) Inventors: Phill Lowe, Tullibody (GB); Christopher John Slevin, Edinburgh (GB); Brian McGuigan, Stirling (GB); Steven Alexander Keatch, Stirling (GB); David Lang, Glasgow (GB); Aman Murtaza Khan, Bedford (GB)

(73) Assignee: LumiraDx UK Ltd., London (GB)

(21) Appl. No.: 18/036,100

(22) PCT Filed: Nov. 10, 2021
(86) PCT No.: PCT/GB2021/052910

§ 371 Date: May 9, 2023

Related U.S. Application Data
(60) Provisional application No. 63/112,074, filed on Nov. 10, 2020.
(60) Provisional application No. 63/120,134, filed on Dec. 1, 2020.
(60) Provisional application No. 63/127,020, filed on Dec. 17, 2020.
(60) Provisional application No. 63/192,450, filed on May 24, 2021.
(60) Provisional application No. 63/112,454, filed on Nov. 11, 2020.
(60) Provisional application No. 63/196,602, filed on Jun. 3, 2021.

(51) Int. Cl. B01L 3/00; B01F 31/65; B01F 33/30; B01F 23/232

(52) U.S. Cl. CPC B01L 3/50273; B01F 31/65; B01F 33/30; B01F 23/232; B01L 2300/14; B01L 2200/16; B01L 2400/0481; B01F 2101/23

(57) ABSTRACT

Described herein is a system and method for determining the presence and/or amount of a target in a sample. In some embodiments, a microfluidic device, such as a strip having a microfluidic network thereon, is inserted into an instrument to perform an assay therein. The strip may be configured to receive the sample from a porous member via capillary action, with minimal or no compression of the porous member. The instrument may include a gas bladder actuation assembly, actuated by an eccentric cam, to compress and decompress a gas bladder on the microfluidic device, thereby enabling fluid movement thereon. The instrument may further comprise a magnet disposed in a fixed location, so as to capture any complexes formed between the target and magnetic particles disposed within the microfluidic device. The magnet may be aligned with an optical detection assembly configured to detect the target captured with the magnetic particles.

### RELATED APPLICATIONS

The present application claims the benefit of and priority to U.S. Patent Application No. 63/112,074, filed Nov. 10, 2020; U.S. Patent Application No. 63/112,454, filed Nov. 11, 2020; U.S. Patent Application No. 63/120,134, filed Dec. 1, 2020; U.S. Patent Application No. 63/127,020, filed Dec. 17, 2020; U.S. Patent Application No. 63/192,450, filed May 24, 2021; and U.S. Patent Application No. 63/196,602, filed Jun. 3, 2021, the entire disclosure of each of which is incorporated herein in its entirety.

Each of the following U.S. patent applications is incorporated herein in its entirety: U.S. Patent Application No. 62/960,421, titled “Fluid Control in Microfluidic Devices”, and filed Jan. 13, 2020, U.S. Patent Application No. 62/972,921, titled “Fluid Control in Microfluidic Devices”, and filed Feb. 11, 2020, and U.S. Patent Application No. 62/991,446, titled “Fluid Control in Microfluidic Devices”, and filed Mar. 18, 2020; U.S. Patent Application No. 63/032,410, titled “Fluid Control in Microfluidic Devices”, and filed May 29, 2020; U.S. Patent Application No. 63/055,744, titled “Fluid Control in Microfluidic Devices”, and filed Jul. 23, 2020; U.S. Patent Application No. 63/067,782 titled “Fluid Control in Microfluidic Devices” and filed Aug. 19, 2020; U.S. Patent Application No. 63/092,371 titled “Fluid Control in Microfluidic Devices” and filed Oct. 15, 2020”; and International Patent Application No. PCT/US2021/013325 (“the '325 Application”) titled “Fluid Control in Microfluidic Devices” and filed Jan. 13, 2021.

### FIELD OF INVENTION

The present invention relates to devices and methods for performing assays.

### BACKGROUND

A microfluidic device, such as a cartridge for example (e.g., a strip), having a microfluidic channel network may be used to perform an assay, e.g., to determine the presence or amount of one or more targets in a sample liquid and/or determine a physiological or physiochemical property of a sample liquid. Such microfluidic devices may be used in conjunction with a diagnostic reader, also referred to as an instrument, which operates the microfluidic device to perform the assay.

### SUMMARY OF THE INVENTION

Disclosed herein, in some aspects, is A system for detection of a target in a liquid sample, comprising: an instrument comprising (i) a microfluidic device introduction port, (ii) a permanent magnet disposed in a fixed, e.g., operatively immovable, position with respect to the introduction port, and (iii) an optical light source disposed in a fixed, e.g., operatively immovable, position with respect to the introduction port; and a microfluidic device received within the instrument via the introduction port, the microfluidic device comprising (i) a microfluidic network disposed therein, wherein the microfluidic network comprises a microfluidic channel in fluid communication with the introduction port, and (ii) a detection zone disposed within the microfluidic network and in fluid communication with the microfluidic channel; wherein, when the microfluidic device is disposed within the instrument, (i) the detection zone experiences a magnetic field emitted by the magnet, the magnetic field sufficient to retain the target within the detection zone if present in the liquid sample passing therethrough, and (ii) the optical light source is configured to illuminate the detection zone, thereby enabling the detection of the target.

In some embodiments, the microfluidic device further comprises: (i) a first reagent disposed within the microfluidic network, the first reagent comprising a first portion configured to bind the target, e.g., a biomolecule indicative of a pathogen, and a second portion comprising a magnetic particle, and (ii) a second reagent disposed within the microfluidic network, the second reagent comprising a first portion configured to bind the target, e.g., forming an immunological sandwich with the first reagent, and a second portion comprising an optically detectable label; wherein the magnetic field is sufficient to retain the first reagent via the magnetic particle. In some embodiments, the retained first reagent is complexed with the target and the second reagent.

In some embodiments, the microfluidic device further comprises a gas bladder configured to move the liquid sample across the microfluidic network. In some embodiments, the gas bladder is configured to move from a compressed configuration to a decompressed configuration, and vice versa. In some embodiments, the instrument further comprises a gas bladder actuation assembly configured to transition the gas bladder from a compressed configuration to a decompressed configuration, and vice versa. In some embodiments, the gas bladder actuation assembly comprises an eccentric cam.

Disclosed herein, in some aspects, is a method, comprising applying a liquid sample held by a porous member to a sample introduction port of a microfluidic device. In some embodiments, the microfluidic device comprises a microfluidic network in fluidic communication with the sample introduction port, and wherein the applying the liquid sample held by the porous member to the sample introduction port comprises drawing at least some of the liquid sample held by the porous member from a tip thereof through the sample introduction port and into at least a portion of the microfluidic network. In some embodiments, the method further comprises performing the drawing the at least some liquid sample by capillary action within the sample introduction port and/or the at least a portion of the microfluidic network. In some embodiments, the drawing the sample through the sample introduction port and into at least a portion of the microfluidic network comprises flowing at least some of the liquid sample drawn from the porous member along at least a portion of the microfluidic network. In some embodiments, the flowing the at least some of the liquid sample drawn from the porous member along at least a portion of the microfluidic network is performed by capillary action. In some embodiments, the microfluidic network includes a capillary stop and the flowing the at least some of the liquid sample drawn from the porous member along at least a portion of the microfluidic network comprises stopping the flowing when a distal liquid-gas interface of the liquid sample reaches the capillary stop, wherein the distal liquid-gas interface comprises an interface between the liquid sample and a gas disposed within the microfluidic device. In some embodiments, the capillary stop comprises a vent in gaseous communication with an ambient atmosphere surrounding the microfluidic device.

In some embodiments, for any method described herein, the method further comprises solubilizing at least one reagent with the liquid sample drawn from the porous member through the sample introduction port and into at least a portion of the microfluidic network, wherein the at least one reagent is disposed within the at least a portion of the microfluidic network. In some embodiments, the at least one reagent comprises a first reagent, the first reagent comprising i) a first portion configured to bind a target indicative of a pathogen, and ii) a second portion comprising a detectable label. In some embodiments, the detectable label comprises an optical detection label. In some embodiments, the detectable label comprises a fluorescent label. In some embodiments, the at least one reagent comprises a second reagent, the second reagent comprising i) a first portion configured to bind the target, e.g., in a sandwich relationship with the target and the first reagent, and ii) a second portion comprising a magnetic particle.

In some embodiments, the method further comprises flowing the at least some liquid including the solubilized at least one reagent along the microfluidic network beyond the capillary stop. In some embodiments, the flowing the at least some liquid including the solubilized at least one reagent along the microfluidic network beyond the capillary stop comprises decreasing a pressure of the gas within the microfluidic device as compared to a pressure of the ambient atmosphere. In some embodiments, the decreasing the pressure of the gas comprises increasing a volume occupied by the gas within the microfluidic network. In some embodiments, the increasing the volume occupied by the gas comprises increasing a height of the microfluidic channel network at a location occupied by the gas within the microfluidic device. In some embodiments, the increasing the height comprises rotating an eccentric member, e.g., a wheel, having a periphery operatively coupled with an exterior of the microfluidic device overlying the location occupied by the gas within the microfluidic device.

In some embodiments, the flowing the at least some liquid including the solubilized at least one reagent along the microfluidic network beyond the capillary stop comprises flowing the at least some liquid including the solubilized reagent through a localized magnetic field. In some embodiments, the flowing the at least some liquid including the solubilized reagent through a localized magnetic field within the microfluidic network comprises retaining at least the second reagent, including second reagent bound to the target, within a detection zone defined by the localized magnetic field. In some embodiments, the method further comprising generating the localized magnetic field using a permanent magnet disposed adjacent the microfluidic device. In some embodiments, the microfluidic device is disposed in an operatively secure state within an instrument comprising the permanent magnet and the permanent magnet is disposed in a fixed, e.g., operatively immovable, position with respect to the microfluidic device when in the operatively secure state. In some embodiments, the magnetic field has a strength of about 500 mT Bz to about 1000 mT Bz, such as about 650 mT Bz to about 850 mT Bz.

In some embodiments, the instrument comprises a light source and the method further comprises i) illuminating the detection zone with light from the light source, and ii) detecting a signal emitted by the detectable label of the first reagent bound to the target and present in the detection zone, wherein the detectable label emits the signal via exposure to the light from the light source. In some embodiments, the method further comprises detecting the presence or amount of the detectable label present in the detection zone and determining the presence or amount of the target present in the liquid sample based on the detected detectable label.

In some embodiments, for any method described herein, the liquid sample comprises a mixture of a liquid buffer and a biological specimen, e.g., a nasal, salivary, throat, nasopharyngeal, mid turbinate, urine, or vaginal specimen, collected from a mammal, e.g., a human being. In some embodiments, the method further comprising, prior to the step of applying the liquid sample, forming the liquid sample by one or more steps including i) receiving a collection swab, the tip of the collection swab having been used to collect the biological specimen, and ii) contacting the tip of the collection swab with the liquid buffer, so as to form the liquid sample. In some embodiments, the method further comprising contacting the porous member with the liquid sample so as to uptake the liquid sample thereon, and thereby enable the applying the liquid sample to the introduction port. In some embodiments, wherein (i) the collection swab is a first collection swab and the porous member is a tip of a second collection swab. In some embodiments, the method further comprising removing the tip of the first collection swab from the liquid sample prior to contacting the porous member with the liquid sample.

In some embodiments, the total volume of the liquid buffer is about 225 microliters or less, 200 microliters or less, about 175 microliters or less, about 150 microliters or less, or about 125 microliters or less. In some embodiments, the total volume of the liquid sample is about 225 microliters or less, 200 microliters or less, about 175 microliters or less, about 150 microliters or less, or about 125 microliters or less. In some embodiments, the liquid buffer comprises a blocking agent, e.g., a protein-based blocking agent, e.g., a protein-based blocking agent such as bovine serum albumin. In some embodiments, the tip of the second collection swab (i) comprises a plurality of fibers, e.g., as a flocked swab tip or a spun fiber swab tip, (ii) comprises a sponge or foam, (iii) is a sintered swab tip, (iv) is a three-dimensional printed swab tip, or (v) includes a combination of two or more swab tips of clauses (i) (iv).

In some embodiments, the step of applying comprises moving the tip of the second collection swab holding the liquid sample from a non-application state to an application state, wherein in the non-application state the liquid sample held by the tip of the second collection swab is not in fluidic communication with the sample introduction port of the microfluidic device and in the application state the liquid sample held by the tip of the second collection swab is in fluidic communication with the sample introduction port. In some embodiments, the liquid sample is formed within a vessel or container. In some embodiments, the vessel or container comprises a sample extraction vial. In some embodiments, the porous member is mechanically and fluidically separated from the microfluidic device during the contacting the liquid sample.

In some embodiments, for any method described herein, the total volume of the liquid sample drawn from the porous member through the sample introduction port and into at least a portion of the microfluidic network is about 6 μl or less, about 5 μl or less, about 4 μl or less, about 3.5 μl or less, or about 3 μl or less.

In some embodiments, for any method described herein, the total volume of the liquid sample drawn from the porous member through the sample introduction port and into at least a portion of the microfluidic network is at least about 1 μl, at least about 2 μl, or at least about 2.5 μl.

In some embodiments, for any method described herein, the method further comprising, after the step of applying, separating the porous member mechanically and/or fluidically from the sample introduction port of the microfluidic device.

In some embodiments, for any method described herein, the applying is performed without substantially compressing the tip of the porous member. In some embodiments, the porous member has a total volume, including the liquid sample, of V immediately prior to the applying the liquid sample to the sample application port and, during the applying, the total volume of the tip remains at least about 0.7×V, at least about 0.8×V, at least about 0.9×V, at least about 0.95×V, or at least about 0.975×V.

In some embodiments, for any method described herein, at least about 75%, at least about 85%, at least about 90%, at least about 95%, at least about 97.5% or essentially all of the liquid sample that enters the microfluidic network from the porous member during the applying is drawn therein by capillary action within the sample introduction port and/or the at least a portion of the microfluidic network.

In some embodiments, for any method described herein, the applying is performed without substantially increasing a pressure experienced by the liquid sample held by the porous member as compared to a pressure experienced by the liquid sample prior to the applying. In some embodiments, the pressure experienced by the liquid sample held by the porous member during the applying is about 1.2×P or less, about 1.15×P or less, about 1.1×P or less, about 1.05×P or less, or essentially the same as P, wherein P is the gas pressure of the ambient atmosphere surrounding the tip of the collection swab.

In some embodiments, for any method described herein, the first collection swab is a nasopharyngeal collection swab, oral swab, oropharyngeal collection swab, or vaginal collection swab. In some embodiments, the liquid sample is formed in a sample extraction tube that comprises an opening through which the tip of the first collection swab is introduced, wherein the method comprises, after the step of forming the liquid sample, obstructing the opening of the extraction tube with the porous member; and wherein the step of applying the liquid sample to the sample introduction port comprises passing the liquid sample from within the extraction tube through the porous member and contacting the sample introduction port with the porous member.

In some embodiments, the method further comprising: after the step of applying the liquid sample, closing the opening of the extraction tube with an impermeable cover. In some embodiments, closing the opening of the extraction tube with the impermeable cover comprises closing the porous member therein.

In some embodiments, for any method described herein, the sample introduction port is disposed at or adjacent a periphery of the microfluidic device. In some embodiments, the sample introduction port is disposed at the periphery of the microfluidic device. In some embodiments, the periphery of the microfluidic device defines a peripheral face and the sample introduction port comprises an opening disposed in the peripheral face. In some embodiments, the sample introduction port is arranged as an end fill introduction port. In some embodiments, the method further comprising determining the presence and/or amount of the target present in the liquid sample that has flowed along the at least a portion of the microfluidic network.

In some embodiments, for any method described herein, the microfluidic network consists essentially of a single microchannel.

In some embodiments, for any method described herein, the method is performed without combining, either concurrently with or subsequently to the step of applying, the liquid sample held by the porous member and applied to the sample introduction port of the microfluidic device with another liquid sample.

In some embodiments, for any method described herein, during the method, the microfluidic device is at least essentially free, e.g., is free, of any liquid other than the liquid sample drawn from the porous member through the sample introduction port and into at least a portion of the microfluidic network.

In some embodiments, for any method described herein, the method is performed without applying a liquid other than the liquid sample held by the porous member to the sample introduction port of the microfluidic device.

In some embodiments, for any method described herein, the method is performed without introducing into the microfluidic device a liquid other than the liquid sample held by the porous member and drawn through the sample introduction port and into at least a portion of the microfluidic network.

In some embodiments, for any method described herein, the microfluidic device lacks any reservoir for a liquid and/or lacks any port for introducing a liquid to the microfluidic device other than the sample introduction port.

In some embodiments, for any method described herein, the microfluidic device comprises an overflow reservoir disposed between the detection zone and a chamber, e.g. a gas bladder, which can be compressed or decompressed to move liquid in the microfluidic device respectively in a proximal or distal direction relative to the sample application port of the microfluidic device.

In some embodiments, for any method described herein, the step of applying is performed after the microfluidic device has been inserted into a microfluidic device introduction port of an instrument configured to operate the microfluidic device to determine the presence and/or amount of a target, e.g., a target indicative of the presence of a pathogen, present in the liquid sample applied to the sample introduction port.

In some embodiments, for any method described herein, the microfluidic network of the microfluidic device is formed of non-porous material.

In some embodiments, for any method described herein, the microfluidic network comprises one or more open channels with minimal or no porosity, such that the microfluidic network is substantially open.

Described herein, in some aspects, is a microfluidic device, comprising: a substrate defining a periphery; and a microfluidic network comprising a sample introduction port and a reagent zone, wherein the sample introduction port extends to and is in fluidic communication with the periphery, and the reagent zone comprises one or more reagents (i) mobilizable by a liquid sample introduced to the reagent zone and (ii) configured to bind to and/or enable the detection of a target indicative of the presence or amount of a pathogen present in the liquid sample introduced to the reagent zone.

Described herein, in some aspects, is a system, comprising: an instrument comprising (i) a microfluidic device introduction port, (ii) a permanent magnet disposed in a fixed, e.g., operatively immovable, position with respect to the introduction port and (iii) an optical light source disposed in a fixed, e.g., operatively immovable, position with respect to the introduction port; and a microfluidic device received within the instrument via the introduction port and disposed in an operatively secured state within the instrument, the microfluidic device comprising (i) a microfluidic network disposed therein, (ii) a first reagent disposed within the microfluidic network, the first reagent comprising a first portion configured to bind a target, e.g., a biomolecule indicative of a pathogen, and a second portion comprising a magnetic particle, (iii) a second reagent disposed within the microfluidic network, the second reagent comprising a first portion configured to bind the target, e.g., forming an immunological sandwich with the first reagent, and a second portion comprising an optically detectable label, and (iv) a detection zone disposed within the microfluidic network; wherein, when the microfluidic device is disposed in the operatively secured state within the instrument (i) the detection zone experiences a magnetic field emitted by the magnet, the magnetic field sufficient to retain the first reagent, e.g., as a sandwich comprising the first reagent, the target, and the second reagent, within the detection zone if present in a liquid solution passing therethrough, and (ii) the optical light source is configured to illuminate the detection zone.

Described herein, in some aspects, is a method, comprising: positioning a microfluidic device with respect to a magnet, wherein the microfluidic device comprises a microfluidic network disposed therein, the microfluidic network comprises at least one mobilizable reagent comprising a magnetic particle, the at least one reagent is configured to bind either directly or indirectly with a target indicative of a pathogen, and the magnet subjects only a portion of the microfluidic network to a magnetic field; introducing a liquid sample into the microfluidic network of the microfluidic device via a sample application port thereof, the liquid sample comprising the target; after positioning the microfluidic device with respect to the magnet: combining at least some of the introduced liquid sample with the at least one mobilizable reagent within the microfluidic network; moving the at least some liquid sample with the at least one mobilizable reagent along the microfluidic network from a first position not subjected to the magnetic field, through the portion of the microfluidic network subjected to the magnetic field, to a second position not subjected to the magnetic field, wherein substantially all of the at least one mobilizable reagent is retained within the portion of the microfluidic network subjected to the magnetic field; and determining the presence and/or amount of the target in the liquid sample based on the retained at least one mobilizable reagent, wherein the steps of combining and moving are performed without substantially modifying either the position of the microfluidic device and magnet with respect to one another or substantially modifying the magnetic field experienced by the portion of the microfluidic network subjected to the magnetic field.

In some embodiments, the steps of introducing, combining, moving, and determining are performed without substantially modifying either the position of the microfluidic device and magnet with respect to one another or substantially modifying the magnetic field experienced by the portion of the microfluidic network subjected to the magnetic field. In some embodiments, the steps of introducing, combining, moving, and determining are performed without modifying either the position of the microfluidic device and magnet with respect to one another or modifying the magnetic field experienced by the portion of the microfluidic network subjected to the magnetic field. In some embodiments, the step of positioning comprises inserting the microfluidic device into a microfluidic device introduction port of an instrument, wherein the instrument comprises the magnet and is configured to operate the microfluidic device to determine the presence and/or amount of the target.

Described herein, in some aspects, is a method, comprising: moving a liquid along a microchannel of a microchannel network of a microfluidic device by increasing or decreasing a pressure of a first gas disposed within the microchannel network and in gaseous communication with a first liquid-gas interface of the liquid; and independently of the increasing or decreasing the pressure of the first gas, oscillating a pressure of a second gas disposed within the microchannel network and in gaseous communication with a second liquid-gas interface of the liquid at a rate and amplitude sufficient to induce mixing of the liquid and a reagent in contact with the liquid within the microchannel.

In some embodiments, the method further comprising performing the increasing or decreasing the pressure of the first gas with a first actuator in contact with the microfluidic device, and performing the oscillating the pressure of the second gas with a second actuator in contact with the microfluidic device. In some embodiments, the first actuator contacts a first location of an exterior surface of the microfluidic device, and the second actuator contacts a second, different location of the exterior surface of the microfluidic device, wherein the first and second locations are spaced apart from one another. In some embodiments, the method further comprising performing one of the steps of (i) increasing or decreasing the pressure of the first gas, and (ii) oscillating the pressure of the second gas, with the respective first or second actuator without simultaneously actuating the other of the first or second actuator.

In some embodiments, the moving the liquid comprises moving the first liquid-gas interface of the liquid along the microchannel over a distance of at least about 1 mm, at least about 1.5. mm, at least about 2 mm, at least about 3 mm, at least about 4 mm, at least about 5 mm, at least about 6 mm, or at least about 7 mm. In some embodiments, the moving the liquid comprises moving the first liquid-gas interface of the liquid along the microchannel over a distance of about 25 mm or less, about 20 mm or less, about 15 mm or less, about 12.5 mm or less, or about 10 mm or less. In some embodiments, the moving the first liquid-gas interface over the distance comprises moving the first liquid-gas interface continuously over the distance. In some embodiments, each of the first and second locations overlies a volume of the microchannel network occupied by the first gas and the second gas respectively and not the liquid. In some embodiments, the first gas and the second gas are the same type of gas.

Described herein, in some aspects, is an extraction vial, comprising: a generally tubular body comprising (i) at a first end, a base, and, at a second, opposing end, an opening; a liquid buffer disposed within the tubular body; and a cap configured to cover the opening, the cap comprising a porous applicator.

Described herein, in some aspects, is a method, comprising: forming a liquid mixture in an extraction vial, the liquid mixture comprising a liquid buffer and a biological specimen; and passing liquid of the liquid mixture through a porous applicator covering an opening of the extraction vial, and applying the liquid directly to a sample application zone of a microfluidic device.

In some embodiments, the forming the liquid mixture comprises introducing a collection tool comprising the biological specimen through an opening of the extraction vial and contacting the liquid buffer disposed therein. In some embodiments, the method further comprising, after the forming the liquid mixture, covering the opening of the extraction vial with an applicator cap, the applicator cap comprising the porous applicator through which the liquid mixture passes to exit the extraction vial. In some embodiments, the microfluidic device comprises a microchannel defined by one or more non-porous impermeable substrates. For example the microchannel may be an open microchannel defined by impermeable walls and without a porous matrix therein through which sample liquid flows.

Described herein, in some aspects, is a method comprising: a) forming an aliquot of liquid sample within a microfluidic channel of a microfluidic device, wherein the aliquot of liquid sample extends from a proximal gas-liquid interface disposed at or adjacent a sample application zone of the microfluidic channel and along the microfluidic channel to a distal liquid-gas interface disposed at a capillary stop zone within the microfluidic channel; b) moving the aliquot of liquid sample including the proximal gas-liquid interface and the distal liquid-gas interface thereof distally along the microfluidic channel until at least a portion of the aliquot of liquid sample contacts one or more mobilizable magnetic reagents disposed at a first location within the microfluidic channel; c) moving the aliquot of liquid sample including the proximal gas-liquid interface and the distal liquid-gas interface thereof and the mobilizable magnetic reagents mobilized thereby distally along the microfluidic channel at least until the proximal gas-liquid interface of the liquid samples is disposed distally with respect to a magnetic reagent capture zone disposed at a second location, distal to the first location, within the microfluidic channel; and d) during the step of moving the aliquot of liquid sample with the mobilized magnet reagents, capturing a mobilized magnetic reagent within the magnetic reagent capture zone.

In some embodiments, at least one of, e.g., both of, the steps of moving the aliquot of liquid sample comprises reducing a ratio Pd/Pp, where Pd is a pressure of the gas acting of the distal liquid-gas interface of the liquid sample and Pp is the pressure of the gas acting on the proximal gas-liquid interface of the liquid sample. In some embodiments, during the process of forming the aliquot of liquid sample, a distal portion of the microfluidic channel disposed distal to the capillary stop zone has a total volume occupied by the gas of the distal liquid-gas interface and at least one of, e.g., both of, the steps of moving the aliquot of liquid sample comprises increasing the total volume occupied by the gas of the distal liquid-gas interface. In some embodiments, increasing the total volume comprises decreasing a force acting upon an exterior surface of the microfluidic device. In some embodiments, the exterior surface is the exterior surface of a wall of the microfluidic channel of the microfluidic device. In some embodiments, decreasing the force comprises rotating an eccentric member in direct or indirect mechanical communication with the exterior surface of the microfluidic device. In some embodiments, the eccentric member is in direct communication with the exterior surface. In some embodiments, the eccentric member is in indirect mechanical communication with the exterior surface via a tensile member tending to exert compressive force on the exterior surface when not engaged with the eccentric member. In some embodiments, the eccentric ember is in indirect mechanical communication with the exterior surface via a lever member that exerts a compressive force on the exterior surface that is positively correlated with a force exerted by the eccentric wheel on the lever member.

In some embodiments, for any method described herein, the gas acting on the proximal gas-liquid interface of the liquid sample is an ambient gas surrounding sample application zone of the microfluidic device. In some embodiments, for any method described herein, the gas acting on the proximal gas-liquid interface of the liquid sample is ambient air.

In some embodiments, for any method described herein, the microfluidic device is an end-fill microfluidic device. In some embodiments, the sample application zone is an opening within a peripheral surface of the microfluidic device.

In some embodiments, for any method described herein, the microfluidic channel defines a total proximal volume between a proximal origin of the sample application zone and the capillary stop zone, and the total aliquot volume of the aliquot of liquid sample is within about 15%, within about 12%, within about 10%, within about 7.5%, within about 5%, within about 2.5%, or about the same as the total proximal volume of the microfluidic channel. In some embodiments, the total proximal volume is at least about 0.5 microliters, at least about 1 microliter, at least about 1.5 microliters, or at least about 2 microliters, or at least about 2.5 microliters. In some embodiments, the total proximal volume is about 10 microliters or less, about 7.5 microliters or less, about 5 microliters or less, about 4 microliters or less, about 3.5 microliters or less, or about 3 microliters or less.

In some embodiments, for any method described herein, the total volume of liquid sample present in the microfluidic device during the method is at least about 0.5 microliters, at least about 1 microliter, at least about 1.5 microliters, or at least about 2 microliters, or at least about 2.5 microliters. In some embodiments, for any method described herein, the total volume of liquid sample present in the microfluidic device during the method is about 10 microliters or less, about 7.5 microliters or less, about 5 microliters or less, about 4 microliters or less, about 3.5 microliters or less, or about 3 microliters or less.

In some embodiments, for any method described herein, the method further comprises, prior to the step of forming the aliquot, disposing the microfluidic device in fixed spatial relationship with a permanent magnet, and wherein the step of forming, both the steps of moving the aliquot of liquid sample, and the capturing are performed without modifying the spatial arrangement of the permanent magnet and the microfluidic device or modifying the magnetic field exerted by the permanent magnet upon the magnetic reagent capture zone.

In some embodiments, for any method described herein, following the step of moving the aliquot of liquid sample including the proximal gas-liquid interface and the distal liquid-gas interface thereof and the mobilized magnetic reagents therein distally along the microfluidic channel, the captured mobilizable magnetic reagents in the magnetic reagent capture zone are surrounded by the gas of the proximal gas-liquid interface of the liquid sample. In some embodiments, the method further comprising detecting the presence of the captured mobilizable magnetic reagents in the magnetic reagent capture zone while the magnetic reagents are surrounded by the gas of the proximal gas-liquid interface of the liquid sample. In some embodiments, the captured magnetic reagents are directly or indirectly labeled with an optical label, and the detecting comprises detecting the present of the optical label. In some embodiments, the optical label is a fluorescent label or a colorimetric label and the detecting comprises detecting an optical fluorescence, an absorbance, and/or a color of the optical label. In some embodiments, the magnetic reagent is the optical label. In some embodiments, the magnetic reagent is indirectly bound to the optical label by a complex including a target to be detected in the liquid sample.

For any method described herein, the liquid sample comprises any liquid sample as described herein (e.g., nasopharyngeal), and may include any buffer solution described herein. For any method described herein, the liquid sample is provided to the sample application zone via any porous member described herein (e.g., applicator, swab, etc.) For any method described herein, the forming of the aliquot may be performed by capillary action. For any method described herein, the microfluidic channel may be open, and/or may comprise impermeable walls. For any method described the reagent may be configured to facilitate detection of a pathogen, for example, detecting a target indicative of the presence of a pathogen, as described herein.

### BRIEF DESCRIPTION OF THE FIGURES

The following Figures (Figs.) are attached to this Specification:

FIG. 1 illustrates an embodiment of a microfluidic device, specifically a microfluidic strip described herein, the microfluidic strip being an end fill strip;

FIG. 2 is a perspective view of a diagnostic reader of the invention;

FIG. 3 is a perspective view of the diagnostic reader of FIG. 2, a microfluidic strip having been inserted into an input port thereof, wherein the microfluidic strip can be any strip described herein (e.g., FIGS. 1, 5a-9b, 17, 18, etc.);

FIG. 4 illustrates a collection swab tip applying sample liquid to a sample introduction port of the end fill strip of FIG. 1;

FIG. 5a is a top planar view of another exemplary microfluidic strip of FIG. 3, the strip having a microfluidic channel network including a sample application port, a microchannel extending from the sample application port, the microchannel including a reagent zone including side cavities to facilitate mixing, a vent stop in gaseous communication with a vent, a measurement zone, and a gas bladder (e.g., an air bladder);

FIG. 5b is a side cross-sectional view of the microfluidic device, specifically a top fill strip of FIG. 5a, further illustrating an arrangement of certain components of the diagnostic reader of FIG. 2 with respect to the strip, the components including a mixing foot (transducer) for transmitting oscillatory energy to sample liquid present within the reagent zone of the microchannel of the strip, a light emitting diode (LED) for performing optical excitation and a photodiode (PD) for performing detection; a bladder foot for compressing and decompressing the air bladder at a distal terminus of the microchannel, a magnet, e.g., a permanent fixed magnet, for retaining magnetic reagents within the measurement zone of the microchannel, and a magnetic shield to reduce the magnetic field experienced by regions of the microchannel that are proximal to the measurement zone;

FIG. 5c is a side cross-sectional view of the microfluidic strip of FIG. 5a, a volume of sample liquid having been applied to a sample application port of the strip;

FIG. 6a is another top planar view of the microfluidic strip of FIG. 5a;

FIG. 6b is a side cross-sectional view of the microfluidic strip of FIG. 6a, further illustrating the components of the reader shown in FIG. 5b, with the bladder actuation foot in a compressed state with respect to the air bladder;

FIG. 7a is another top planar view of the microfluidic strip of FIG. 5a, with an amount of sample liquid having been applied to the sample application port of the strip and proceeded along a microchannel of the strip until a distal liquid-gas interface of the sample liquid reaches a capillary vent stop of the microchannel;

FIG. 7b is a side cross-sectional view of the microfluidic strip of FIG. 7a, with the sample liquid in the state shown in FIG. 7a and the components of the reader in the state shown in FIG. 6b;

FIG. 8a is another top planar view of the microfluidic strip of FIG. 7a;

FIG. 8b is a side cross-sectional view of the microfluidic strip of FIG. 8b, with the sample liquid in the state shown in FIG. 7a and the components of the reader in the state shown in FIG. 6b except that the mixing foot (transducer) of the reader is shown in an oscillating state to facilitate mixing of the sample with reagents deposited within the reagent zone of the microchannel of the strip;

FIG. 9a is another top planar view of the microfluidic strip of FIG. 5a with the sample liquid having been drawn along the microchannel of the strip until the distal liquid-gas interface of the sample enters a gas bladder disposed at a distal terminus of the microchannel;

FIG. 9b is a side cross-sectional view of the microfluidic strip of FIG. 9a, with the sample liquid in the state shown in FIG. 9a and the components of the reader in the state shown in FIG. 6b except that FIG. 9b illustrates an actuation motion of the bladder actuation foot, retracting from the gas bladder, allowing the upper wall of the gas bladder, which had been placed under tension by the bladder actuation foot, to expand upwards thereby increasing the volume of the gas bladder and decreasing the gas pressure therein to draw the sample liquid along the microchannel to establish the sample liquid state shown in FIG. 9a;

FIG. 10 illustrates fluorescent images of a detection zone of respective end fill strips fabricated as the end fill strip of FIG. 1 and operated using standard solutions of SARS-CoV/SARS-CoV-2 antigen;

FIG. 11a is a perspective view of a diagnostic reader (such as from FIGS. 2-3) including the mechanism for actuating the bladder foot (actuation foot) and a microfluidic strip having been inserted into an input port of the reader;

FIG. 11b is a perspective view of the microfluidic strip of FIG. 11b showing engagement between a notch of the strip and an engagement arm of the diagnostic reader;

FIG. 11c is a cutaway side partial cross-sectional view of the reader and strip of FIG. 11a;

FIG. 11d is a cutaway front partial cross-sectional view of the reader of FIG. 11a illustrating the magnet and photodiode, the strip having been removed;

FIG. 11e is a perspective partial cutaway view of the reader showing the magnet, shield, photodiode and LED;

FIG. 11f is a perspective view of the strip of FIG. 11a showing engagement of the strip with the engagement arm of FIG. 11b and a mixing foot (transducer) of the reader;

FIG. 12a illustrates a third embodiment of an end fill microfluidic strip of the invention, the end fill strip having an overfill reservoir;

FIG. 12b illustrates a second embodiment of an end fill microfluidic strip of the invention, the end fill strip having a tapered transition zone entering a gas bladder thereof,

FIG. 13a illustrates a variation of the end fill microfluidic strip of FIG. 12a, wherein the capillary stop is located proximal to the reagent zone.

FIG. 13b illustrates a variation of the end fill microfluidic strip of FIG. 12b, wherein the capillary stop is located proximal to the reagent zone.

FIG. 14a depicts an exemplary shape and design for a magnet and spacer for use with a microfluidic strip described herein;

FIG. 14b depicts another exemplary shape and design for a magnet and spacer for use with a microfluidic strip described herein;

FIG. 14c depicts another exemplary shape and design for a magnet and spacer for use with a microfluidic strip described herein;

FIG. 14d depicts another exemplary shape and design for a magnet for use with a microfluidic strip described herein;

FIG. 14e depicts another exemplary shape and design for a magnet for use with a microfluidic strip described herein;

FIG. 15 depicts a perspective view of an exemplary gas bladder actuator of the invention;

FIG. 16 is a partial perspective view of a first embodiment of a mechanism for actuating a bladder actuation foot to perform the liquid manipulation functions as described for a microfluidic strip described herein (e.g., FIGS. 1, 6b, 7b, 8b, 9b, 17, and 18);

FIG. 17 illustrates, a top view of a top fill microfluidic device as disclosed in the '325 Application;

FIG. 18 illustrates, a bottom view of the top fill microfluidic device of FIG. 17

FIG. 19 illustrates an exemplary top view of the top fill microfluidic device of FIG. 17 with the proximal portion of the microfluidic device removed such that a sample application port is formed in a peripheral face of the microfluidic device;

FIG. 20 illustrates a bottom view of the microfluidic device of FIG. 19;

FIG. 21a illustrates a first data plot of fluorescence against target concentration for target detection using a top fill strip and an end fill strip; FIG. 21b illustrates a second data plot of fluorescence against target concentration for target detection using a top fill strip and an end fill strip;

FIG. 21c illustrates a sensitivity measurement for the second data plot;

FIG. 22 illustrates an embodiment of an extraction vial having a porous sample applicator for applying a liquid sample directly to a microfluidic strip;

FIG. 23 illustrates a second embodiment of an extraction vial having a porous sample applicator for applying a sample directly to a microfluidic strip;

FIG. 24a illustrates an exploded view of a third embodiment of an extraction vial, the extraction vial including (i) an applicator cap having an integral collection swab for collecting a biological specimen and a porous sample applicator for applying a sample directly to a microfluidic strip and (ii) a tubular body containing a liquid buffer and sealed with a seal;

FIG. 24b illustrates the applicator cap of the extraction vial of FIG. 24a with a sealing cap of the application cap having been removed therefrom;

FIG. 24c illustrates the applicator cap of the extraction vial of FIG. 24a with the sealing cap having been removed and a tip of the collection swab holding a biological specimen;

FIG. 24d illustrates the application cap of FIG. 24c with the seal of the tubular body removed and the integral collection swab inserted into the liquid buffer within the tubular body to prepare a liquid mixture of the liquid buffer and biological specimen;

FIG. 24e illustrates the extraction vial of FIG. 24d with the extraction vial positioned in an application orientation;

FIG. 24f illustrates the extraction vial of FIG. 24e with the sealing cap of the application cap having been removed;

FIG. 24g illustrates the use of the extraction vial of FIG. 24f to apply a filtrate liquid of the liquid mixture to a sample application zone of a microfluidic device disposed in an operational position of a diagnostic reader;

FIG. 25a illustrates an embodiment of an extraction vial having a porous sample applicator for applying a liquid sample directly to a microfluidic strip;

FIG. 25b illustrates an exploded view of the extraction vial of FIG. 25a showing the tubular body, applicator cap, and sealing cap;

FIG. 25c illustrates an exploded perspective view of the applicator cap of the extraction vial of FIG. 25a with the porous applicator having been removed from the remainder of the applicator cap;

FIG. 25d illustrates an exploded side view of the applicator cap of the extraction vial of FIG. 25a, with an external textured surface of the cap (see FIG. 25c) not shown and illustrating internal features of the cap and porous applicator;

FIG. 26a illustrates a perspective view of an applicator cap with porous applicator suitable for use with, e.g., the extraction vial of FIG. 25a; and

FIG. 26b illustrates an exploded view of the applicator cap with porous applicator of FIG. 26a.

### DETAILED DESCRIPTION

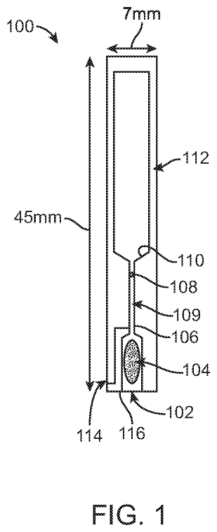

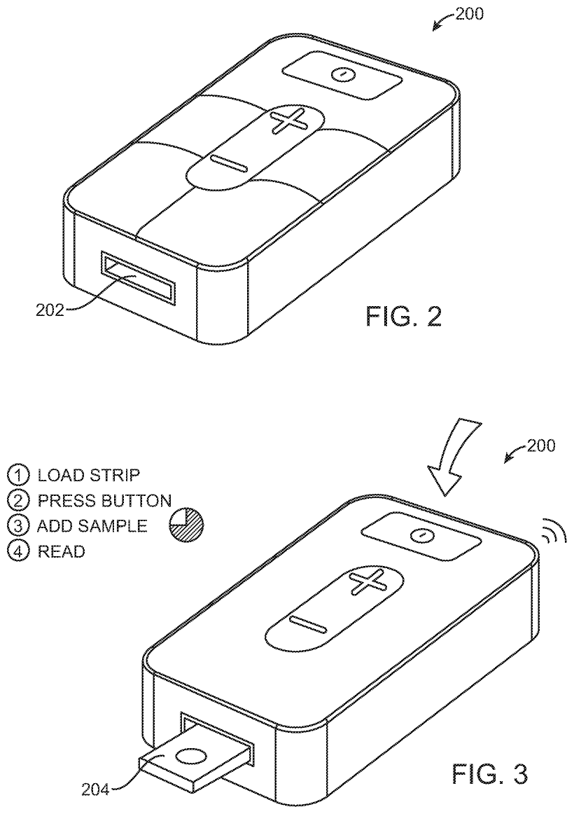

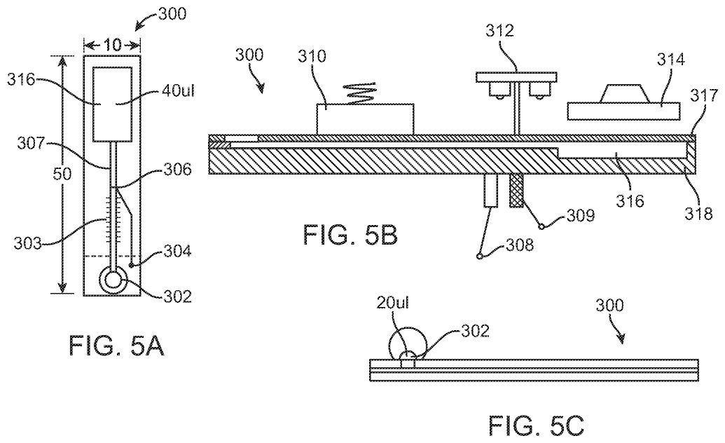

FIG. 1 depicts an exemplary top view of an embodiment of a microfluidic device, which is an end fill diagnostic strip 100. As used herein the term “microfluidic device” may be used interchangeably with the term “microfluidic strip”, “diagnostic strip”, and/or “cartridge”. As described herein, the microfluidic device depicted in FIG. 1 is an end fill strip. In some cases, for any embodiment described herein, the microfluidic device can be a top fill strip (see FIGS. 5a-9b for example). The diagnostic strip (end fill strip) 100 may be operated by a diagnostic reader, such as the diagnostic reader 200 shown in FIGS. 2 and 3. As used herein, the term “diagnostic reader” may be used interchangeably with the term “instrument”. The diagnostic reader 200 operates the end fill strip (for example the end fill strip 100 in FIG. 1) to determine the presence and/or amount of at least one target (e.g., a biomolecule such as a protein, antigen, and/or antibody) present in a sample liquid applied to the end fill strip. The end fill strip 100 may be formed of materials and layers similar to those disclosed for the microfluidic strip disclosed in the '325 Application with reference character 10 (for example, see FIG. 1 of the '325 Application). For example, the end fill strip 100 includes an upper substrate and a lower substrate each composed of 100 μm thick polyester film. A lower surface of the upper substrate and an upper surface of the lower substrate are adhered in opposition by an adhesive layer, 110 μm thick. The adhesive layer occupies less than all of the area of the surfaces between the upper and lower substrates to define a microfluidic channel network therebetween.

With reference to FIG. 1, proceeding from a proximal periphery of the end fill strip, the microfluidic network includes a sample introduction port 102, a reagent zone 104, a capillary stop 106, a detection channel 109 including a detection zone 108, a transition zone 110, and a gas bladder 112. A vent channel 114 includes a first opening disposed within the microfluidic network at the capillary stop and a second opening disposed adjacent a lateral periphery of the end fill strip. As used herein, the term “sample introduction port” may be used interchangeably with the terms “sample introduction zone”, “sample application zone”, or “sample application port”. As described herein, the reagent zone may store reagents corresponding to a target desired for detection. For example, the reagents in the reagent zone may correspond to COVID-19 detection, and/or reagents that correspond to SARS-CoV-2 antigen detection.

In some embodiments, the capillary stop 106 is located proximal to the reagent zone 104. Accordingly, in such embodiments, application of the liquid sample to the sample introduction port 102 enables the liquid to flow up to the capillary stop, prior to entering the reagent zone. In some embodiments, the capillary stop is positioned so as to allow a metered, predetermined amount of the liquid sample to be assayed (for e.g., via a diagnostic reader as described herein). As described herein, the liquid sample may then be moved to a first position to allow the liquid sample enter the reagent zone 104, where the liquid sample may be allowed to interact and mix with reagents located therein. The sample may then move to and/or through the detection zone for detection of a target found within the liquid sample (as described herein).

As with strip of the '325 Application (with reference character 10), the microfluidic channel network of the end fill strip 100 has side walls defined by the adhesive layer disposed between the upper and lower substrates, an upper wall defined by those portions of the upper substrate unoccupied by the adhesive layer, e.g., overlying absent portions of the adhesive layer, and a lower wall defined by those portions of the lower substrate unoccupied by adhesive layer, e.g., underlying the absent portions of adhesive layer. The upper and lower substrates and adhesive layer are non-porous and impermeable to liquid samples. The channel of the microfluidic network is open, e.g., not filled with or substantially occupied by, e.g., is free of, a solid phase or porous membrane through which liquid must pass.

The microfluidic network has a length axis parallel to the length of the end fill strip, a width axis that is perpendicular to the length of the end fill strip and parallel to the primary plane defined by the end fill strip, and a height axis that is perpendicular to the length of the end fill strip and perpendicular to the primary plane defined by the end fill strip. The end fill strip 100 has a length along the length axis between the proximal periphery 116 and opposed distal periphery of 45 mm and a width along the length axis between the opposed lateral peripheries of 7 mm. The microfluidic network has a height along the height axis of 110 μm. The sample application zone 102 and reagent zone 104 have a width of 2.6 mm taken along the width axis. The reagent zone 104 has a length of 7 mm taken along the length axis. The detection channel 109 has a width of 0.65 mm taken along the width axis and a length of 10 mm taken along the length axis. The transition zone 110 progresses from the width of the detection channel to the width of the gas bladder 112 along a length of about 1.5 mm. The gas bladder 112 has a width of 4.8 mm taken along the width axis and a length (not including the transition zone) of 24 mm taken along the length axis.

The sample application zone is a port 102 (opening) extending into the microfluidic network through the proximal periphery 116 of the end fill strip. The sample application zone defines a proximal origin of the microfluidic channel network and places the microfluidic channel network in gaseous communication with a gas, e.g., air, of the ambient atmosphere surrounding the end fill strip (for example, as described for strip 10 in the '325 application). A distal end of the gas bladder 112 defines the distal terminus of the microfluidic channel network. The first opening of the vent channel 114 places the capillary stop in gaseous communication with the ambient atmosphere. Except for the first opening of the vent channel, portions of the microfluidic network disposed distally to the sample application zone are sealed with respect to the ambient atmosphere and other sources of gas external to the microfluidic network.

The reagent zone of the end fill strip 100 includes first and second mobilizable reagents for the determination of SARS-CoV-2 antigen, e.g., the SARS-CoV/SARS-CoV-2 Nucleocapsid Antibody, Mouse MAb-Latex reagent and the SARS-CoV/SARS-CoV-2 Nucleocapsid Antibody, Rabbit Mab-Mag Ag reagent described in Example 2 of the '325 Application.

Prior to the introduction of the liquid sample into the microfluidic network of the end fill strip, the reagents therein are in a dry state, e.g., a lyophilized state or other state in which the moisture content is insufficient to cause the reagents to flow as a liquid. The end fill strip lacks pre-stored liquid reagents, e.g., lacks a reservoir with liquid stored therein.

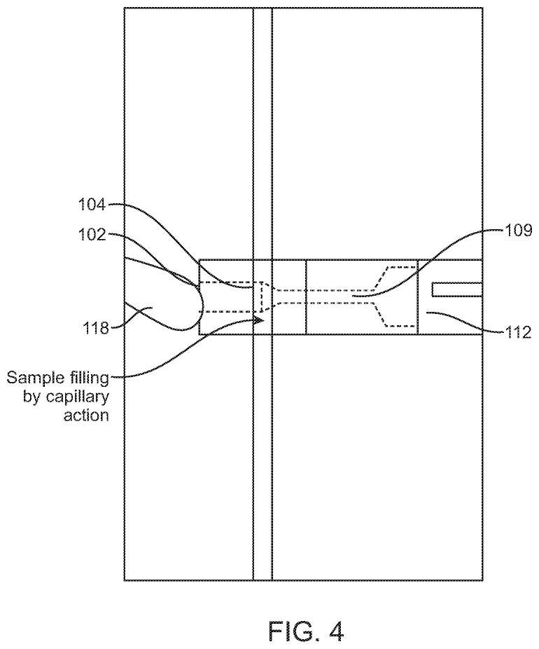

With reference to the FIG. 4, a liquid sample may be applied directly to the sample application port 102 of the end fill strip 100 using a collection tip of a swab 118. Liquid sample applied to the sample application port 102 is drawn by capillary action from the tip of the collection swab through the sample introduction and into the microfluidic network of the end fill strip 100. During the application, the tip of the collection swab 118 remains substantially uncompressed, e.g., the tip is not substantially compressed to reduce the volume of the tip and “squeeze” liquid sample from the tip or to force liquid sample into the microfluidic network via the application of external pressure. Instead, for example, the liquid sample is drawn from the swab 118 at least partially via capillary action (as described herein).

In some embodiments, the liquid sample flows by capillary action into the reagent zone until a distal liquid-gas interface of the flowing liquid sample reaches the capillary stop, at which point the liquid sample ceases to flow. In other embodiments, the capillary stop is located proximal to the reagent zone, and thus liquid sample flows by capillary action until a distal liquid-gas interface of the flowing liquid sample reaches the capillary stop, at which point the liquid sample ceases to flow, before reaching the reagent zone. Exemplary liquid samples include a nasal, salivary, throat, mid turbinate, nasopharyngeal, urine, or vaginal sample collected from a mammal, e.g., a human being. The liquid sample may further include, e.g., be a mixture including, an additional liquid, such as a buffer. The liquid sample may alternatively be applied to the sample application zone using an applicator other than a collection swab tip. For example, sample may be applied using a fibrous applicator, sponge applicator, or foam applicator that are not a tip of a collection swab. The applicator may be a porous applicator of an extraction vial as disclosed herein. An oral fluid applicator such as an adsorbent pad may be used for the collection and application of oral fluid samples, such as salivary samples, to the sample application port. In some embodiments, the collection swab tip that is used to apply the liquid sample to the end fill strip is also used to collect the biological specimen and form the liquid sample. In other embodiments, the collection swab tip that is used to apply the liquid sample to the end fill strip is a different collection swab tip than used to collect the biological specimen. For example, the end fill strip may be a component of a kit including a first collection swab used to collect a biological specimen and prepare a liquid sample including the biological specimen and a liquid buffer, and a second collection swab used to apply the liquid sample to the application zone of the end fill strip.

Once a sufficient amount of liquid sample has been drawn from the collection swab tip or other applicator (e.g., other porous member) into the microfluidic network and prior to performing an analysis on such liquid sample using the microfluidic device, the collection swab tip or other applicator is typically removed from the sample application port. For example, with reference to FIG. 4, the collection swab 118 tip is removed from the sample application port after the liquid sample is drawn into the microfluidic network so that the collection swab tip (and swab 118) are not in fluid or mechanical communication with the microfluidic device (end fill strip). Subsequently, the end fill strip is operated by the diagnostic reader, e.g., to determine the presence or absence of a target in the liquid sample. After use to apply the liquid sample, the collection swab tip or other applicator may be discarded or preserved to permit further analysis of the remaining liquid sample thereon. In some embodiments, wherein the capillary stop is located proximal to the reagent zone (i.e., upstream of the reagent zone), the sample liquid fills the microfluidic network up to the capillary stop upon being applied, therefore providing a metered, predetermined volume of the liquid sample received from the porous member, e.g., collection swab.

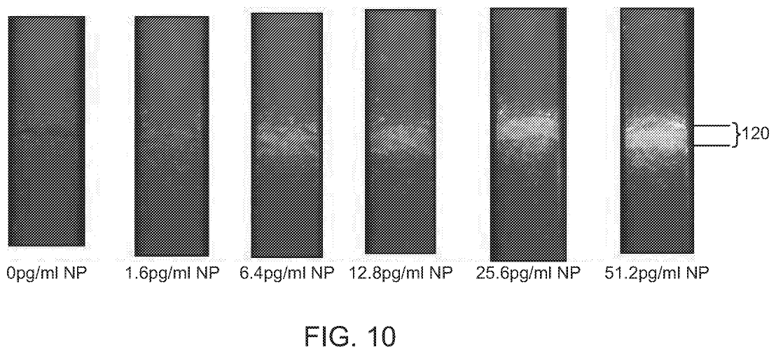

In use, the end fill strip may be operated as follows. The end fill strip 204 (for example, end fill strip 100 described herein, or any other microfluidic strip described herein) is inserted into the microfluidic device introduction port 202 of a diagnostic reader 200 as shown in attached FIGS. 2 and 3. In some cases, the end fill strip is inserted within the diagnostic reader until reaching an operatively secured state. Insertion of the end fill strip within the diagnostic reader (for example, into an operatively secured state) allows the detection zone of the end fill strip to be placed in proximity to a permanent magnet within the diagnostic reader. For example, FIGS. 5A-9B depict other exemplary embodiment of a microfluidic strip 300, wherein a permanent magnet 309 is depicted on a side of the lower substrate opposite to the microfluidic network. The permanent magnet can be similarly positioned for other microfluidic strips described herein, including microfluidic strip 100. The magnet is fixed, e.g., operatively immovable, with respect to the detection zone of the inserted end fill strip (for example, 100, 300). Accordingly, the magnet does not need to be displaced or moved in order for the detection zone to experience a magnetic field, nor does the magnet need to be moved to capture reagent particles and/or move reagent particles along a microfluidic strip. Instead, the magnet can be configured to enable the collection of reagent particles at high concentration and at a tightly defined, fixed position on the microfluidic strip (for example, see FIG. 10, reference character 120). The tightly defined position can be aligned with an optical detector (e.g., photodiode) on the diagnostic reader for detection of a target in a liquid sample.

The magnet may comprise of materials common to high strength permanent magnets, such as neodymium and/or other rare earth elements. In some embodiments, the magnet can provide a magnetic field strength experienced in the detection channel (and more specifically, the detection zone located within the detection channel) of at least about 500 mT Bz, about 600 mT Bz, 700 mT Bz, 750 mT Bz, 775 mT Bz, 800 mT Bz, or 900 mT Bz. In some embodiments, the magnet can provide a field strength experienced in the detection channel (and thus detection zone) from about 500 mT Bz to about 1000 mT Bz. In some embodiments, the magnet can provide a field strength experienced in the detection channel (and thus detection zone) from about 650 mT Bz to about 850 mT Bz.

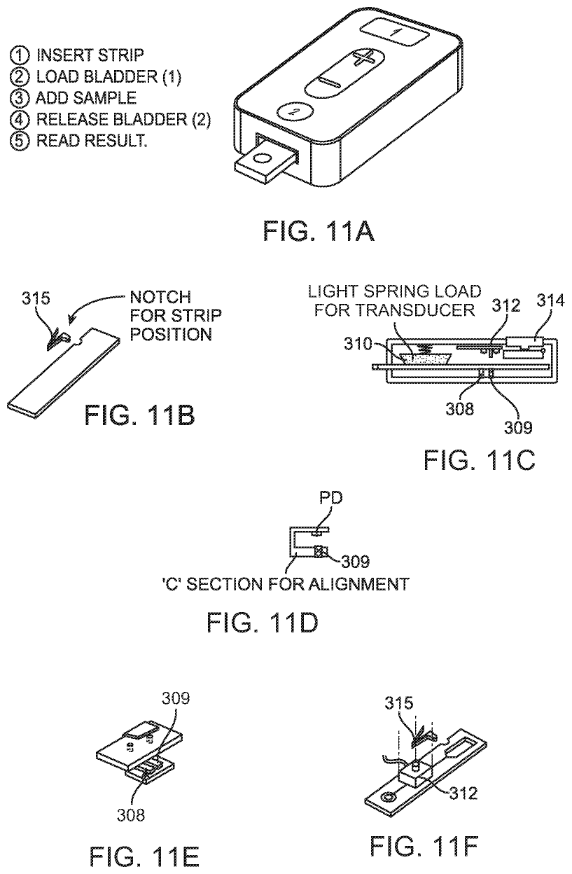

The magnet can be of any shape configured to provide a minimum magnetic field strength (as described herein) to a microfluidic strip within a diagnostic reader, as described herein. In some embodiments, the magnet is positioned (for example, within the diagnostic reader) with a length that extends across the width of the microfluidic strip. As described herein, the magnet can be configured to enable the collection of reagent particles at a tightly defined, fixed position on the microfluidic strip, and thus a width of the magnet can be limited to a prescribed amount (for example, limited to a length of about a detection zone, such as the length 120 shown in FIG. 10). In some embodiments, the magnet comprises a rectangular shape. In some embodiments, the rectangular shape has dimensions of 12.5 mm (length)×4.0 mm (width)×1.0 mm (thickness)—wherein the length of the magnet extends across a width of the microfluidic device (see FIG. 11 (e) for example. In some embodiments, the magnet is polarized with the north and south face on the flat sides of the magnets (as opposed to the edges). The magnet can be configured to touch the strip within the diagnostic reader, and thereby collect the reagent particles at and/or about the location aligned with where the magnet touches the strip. In some embodiments, the magnet comprises high magnetic permittivity (for example, to effectively attract nearby field lines to the shield material).

In some embodiments, the magnet is polarized so that the north pole of the magnet is facing the front of the instrument. Thus, in some cases, the north pole is on the large flat face, not the long edge or short edge. The south pole is on the opposite face.

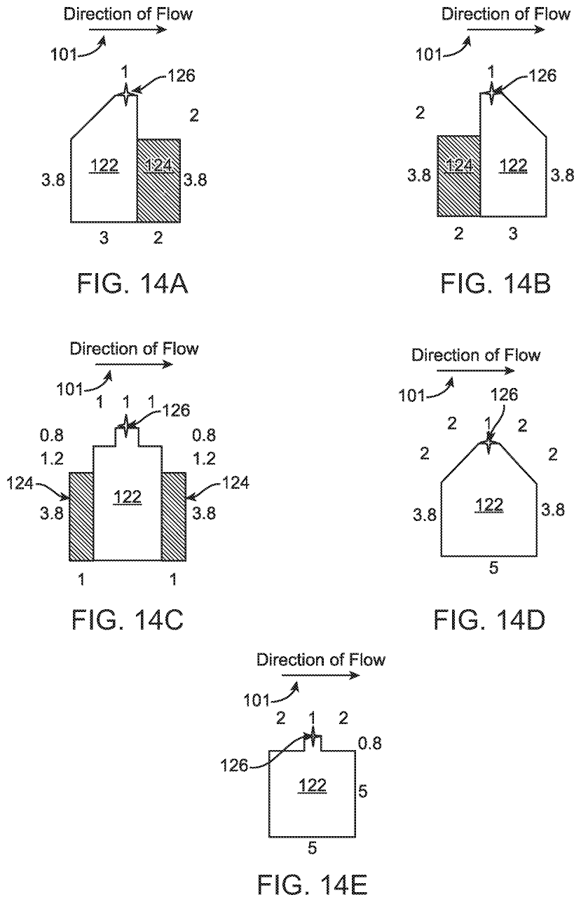

FIGS. 14a-14e depict other exemplary shapes and designs for a magnet (122) described herein with reference to the flow of sample liquid on a microfluidic strip (described herein) from a proximal to distal periphery. FIGS. 14a-14e further depict at least one spacer 124 with the magnet, wherein the shield can be positioned on a distal side of the magnet (FIG. 14a), on a proximal side of the magnet (FIG. 14b), or on both the proximal and distal sides of the magnet (FIG. 14c). In some embodiments, spacer (124) is an optional element that can serve to provide a uniform structure (relating to the magnet) for fitting in to the diagnostic reader. It may typically be made from a non-ferrous material e.g., a polymer and does not participate actively in generation or control of the magnetic field. Each of FIGS. 14a-14e depict exemplary dimensions (in mm) for the width and thickness for each magnet and shield depicted. For example, in FIG. 14a, the magnet 122 is depicted with a bottom width of 3 mm, and proximal side thickness of 3.8 mm. For each magnet 122 and shield 124 depicted in FIGS. 14a-14e, the respective length as extending across the width of the microfluidic strip (as described herein, also see FIG. 11(e) for example) is about 15 mm (for example, the length will be seen as pointing into the page). The optical center point 126 (for example, within an optical window of the diagnostic reader) is also depicted with reference to the magnet and where it touches the microfluidic strip. For example, by aligning the optical center point 126 and the magnet 122, the magnetic field experienced in the detection zone can maximize collection of reagent magnetic particles that will be aligned with a light source and detector for detection (e.g., LED and a photodiode, as described herein). In some embodiments, the optical window of the diagnostic reader is about 2 mm along a length of the microfluidic strip (e.g., along a length of the detection channel as described herein), and about 2.4 mm across the width of the microfluidic strip (e.g., across a width of the detection channel).

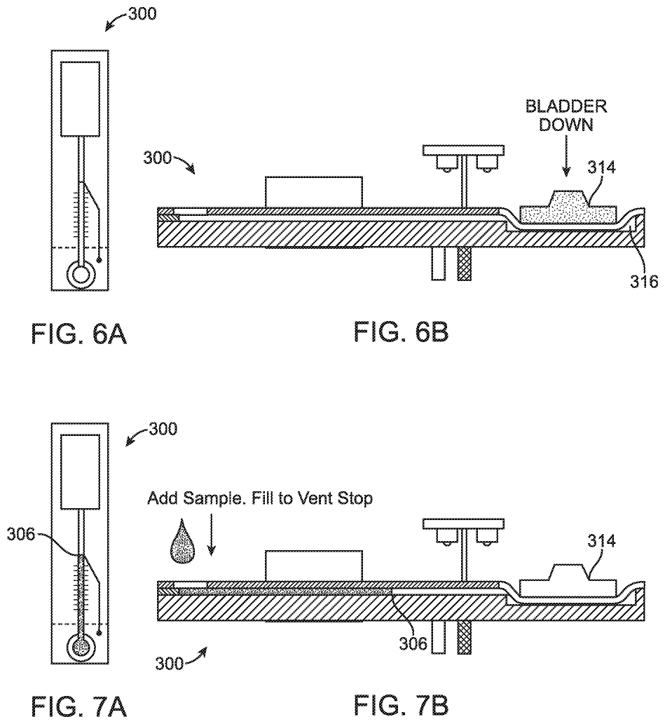

Prior to application of the sample, the gas bladder (as described herein for any microfluidic strip) is compressed, e.g., as shown in attached FIG. 6b and as disclosed in the '325 application with respect to the operation of strip 10 and reader 111. Liquid sample is applied to the sample application port (sample introduction port), e.g., see FIG. 4. Some of the liquid sample flows by capillary action from the collection swab 118 tip through the sample application 102 port and into the microfluidic network including through the reagent zone until the distal liquid-gas interface of the liquid sample reaches the capillary stop. As described herein, in other embodiments, wherein the capillary stop is located proximal to the reagent zone (i.e., upstream of the reagent zone), the liquid sample will stop flowing upon reaching the capillary stop (before reaching the gas bladder). During use, the liquid sample applied to the sample application port 102 via the collection swab 118 tip may be the only liquid introduced to the microfluidic network of the end fill strip. As described herein, other means for delivering the liquid sample to the end fill strip can be used (e.g., vial).

After the sample liquid has reached the capillary stop and ceased flowing, the sample liquid within the reagent zone is allowed to incubate in the presence of the reagents disposed therein (as described herein). The reagents may include magnetic particle reagents disposed within the reagent zone. In some cases, the magnetic particle reagents, when moved with the liquid sample to the detection zone (as described herein) are used with the permanent magnet to immobilize reagent and target molecules, as described herein. For example, in some embodiments, a first reagent disposed within the reagent zone comprises i) a first portion configured to bind the target of the liquid sample, e.g., a biomolecule indicative of a pathogen, and ii) a second portion comprising a magnetic particle. In some embodiments, a second reagent disposed within the reagent zone comprises i) a first portion configured to bind the target, e.g., forming an immunological sandwich with the first reagent, and ii) a second portion comprising an optically detectable label.

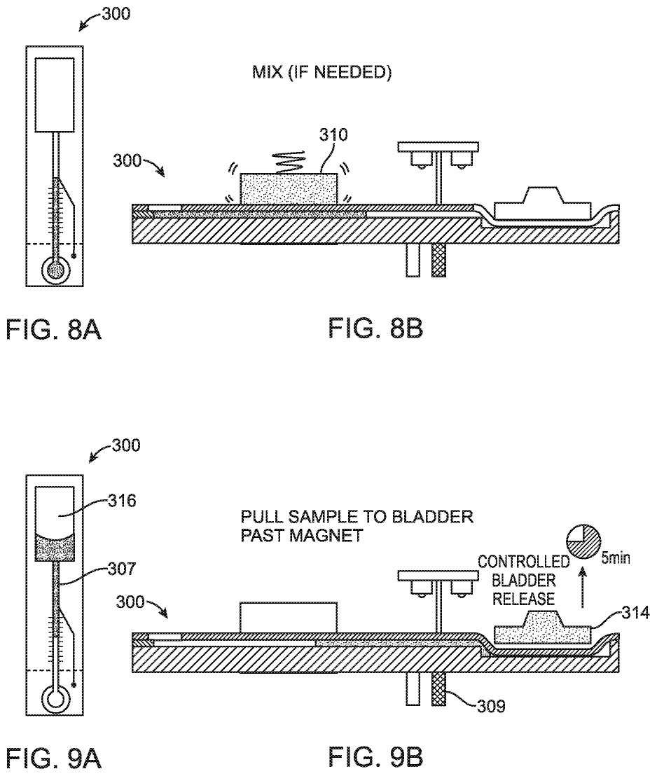

As described herein, the reagents may be in a dry pre-assay state (for example within the reagent zone) and may be solubilized with the liquid sample via contact thereto. In the presence of a target, the mobilized reagents form a complex with the target, e.g., as described in Example 2 of the '325 Application. Incubation may be maintained for a period of time suitable to mobilize a sufficient amount of reagents to facilitate detection, e.g., within about 15 min or less, about 12.5 min or less, about 10 min or less, about 7.5 min or less, or about 5 min or less beginning with the time the sample was applied to the sample application port and ending with the time at which a result of the detection is determined. During the incubation period, the sample liquid within the reagent zone may be oscillated to facilitate mixing, e.g., as shown in attached FIG. 8b or as disclosed in the '325 Application for strip 10 and reader 111.

In other embodiments, wherein the capillary stop is located proximal to the reagent zone, the liquid will move to another point in the microfluidic network correlating to the reagent zone. Accordingly, as described herein, the liquid sample will then incubate with the reagents, including the magnetic particle reagents. The liquid sample may be moved from the capillary stop to the reagent zone via partial decompression of the gas bladder (as described herein).

Once the incubation period has finished, the liquid sample with mobilized reagents is drawn further along the microfluidic network through the detection channel, past the detection zone, e.g., by decompressing the gas bladder as shown in attached FIG. 9b or as described in the '325 application with respect to strip 10 and reader 111. For example, the liquid sample, e.g., essentially all of the liquid sample that was added to the microfluidic device, may be drawn distally along the microfluidic network until a proximal gas-liquid interface of the liquid sample has moved distally beyond the detection zone leaving the captured magnetic particle reagent surrounded by the gas of the proximal gas-liquid interface. Typically, the gas of the proximal gas-liquid interface is the ambient gas, e.g., air, surrounding the microfluidic device. This process optionally includes drawing some of the liquid sample into the gas bladder. As the mobilized magnetic particle reagent (as described herein) enters the detection zone, the magnetic particle reagent, including any reagent complexed with the target and detectable label reagent, is subjected to the magnetic field exerted by the permanent magnet and retained therein. As can be seen from FIG. 10, substantially all of the magnetic particle reagent is primarily retained (shown as concentration of nucleoprotein in pico gram/milliliter) within an area (the detection zone, e.g., 108 in FIG. 1) having a length 120 along the longitudinal axis of the microfluidic network (channel) that is small compared to the overall length of the network. Detectable label reagent that is not complexed with the target and magnetic reagent is not retained within the detection zone. Once sample has flowed past the detection zone, the gas bladder is recompressed to remove the sample liquid and any unretained reagents (e.g., uncomplexed detectable label) from the detection zone by moving the sample liquid proximally toward the sample application zone (e.g., 102 in FIG. 1) of the strip, e.g., as described in the '325 Application for strip 10 and reader 111. As an alternative to recompressing the gas bladder to remove sample liquid from the detection zone, the reader may continue decompressing the gas bladder until a proximal gas-liquid interface of the sample liquid has been moved distally through and beyond the detection zone. In either case, the detectable label complexed with the magnetic particle and retained within the detection zone is indicative of the presence and/or amount of detectable label present in the liquid sample applied to the strip. The complexed detectable label can be detected using, for example, optical or electrochemical techniques. Optical techniques include colorimetry and fluorescence, which may be detected using a device, such as a photodetector, or without a device and directly by eye. FIG. 10 illustrates fluorescent images of the detection zone of the end fill strip for concentrations of SARS-CoV/SARS-CoV-2 antigen using the aforementioned reagents of Example 2 of the '325 Application. A concentration of 1.6 pg/ml of the nucleoprotein is clearly detectable above the blank of 0 pg/ml.

In some other embodiments, for any microfluidic device described herein, prior to performing the assay, the magnetic reagent particles are disposed within the detection zone, wherein movement of the liquid sample through the detection zone enables a target (within the liquid sample, as described herein) complexed with a reagent particle within the reagent zone to be captured by the magnetic reagent particle (for example, the magnetic reagent particle may be configured to form a complex with the target and/or reagent particle from the reagent zone). The magnetic reagent particle may be held in place via the magnet in the diagnostic reader, as described herein.

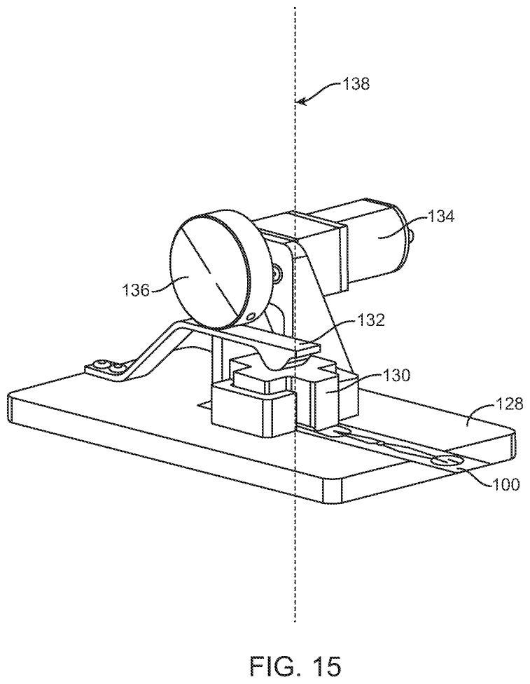

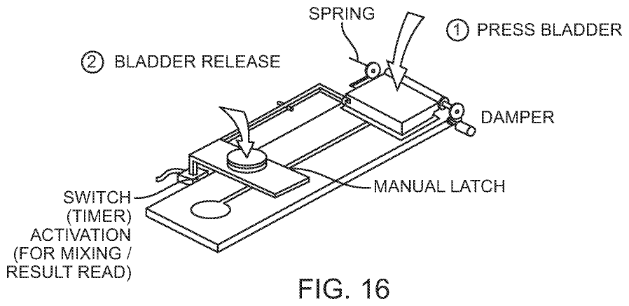

The '325 application describes methods and components for compressing and decompressing the gas bladder (for example, using a piezoelectrically driven actuator). FIG. 16 depicts another exemplary assembly for compressing and decompressing a gas bladder for a microfluidic strip described herein. FIG. 15 depicts a perspective view of an actuator using an eccentric cam (eccentric wheel) for actuating the compression and decompression of a gas bladder within an instrument (e.g., diagnostic reader) to move liquid sample within the microfluidic network of a microfluidic device 100 (e.g., end fill strip) as disclosed herein. The actuator includes a base 128, which supports a microfluidic device 100 in an operatively secured position. The base 128 also supports a motor 134, which includes an eccentric wheel 136 (eccentric cam) secured to a rotatable shaft driven by the motor 134. A periphery of the eccentric wheel contacts a central portion of a lever arm 132, which has a first end fixed to the base 128 and a second, free end, overlying an upper surface of an actuation foot 130. The actuation foot 130 includes a lower surface overlying the gas bladder of the microfluidic device 100. The actuation foot 130 is free to move along a vertical axis 138 with respect to the microfluidic device 100 but is otherwise constrained. As the motor 134 rotates the eccentric wheel 136, periphery drives the lever arm up or down along the vertical axis 138. The free end of the lever arm 132 drives the actuation foot 130 up or down along the vertical axis 138 against the upper surface of the gas bladder. The vertical movement compresses or decompresses the gas bladder permitting liquid sample within the microfluidic network of the microfluidic device 100 to be moved therealong as described above and in the '325 Application.

In some embodiments, a variation of the gas bladder actuation assembly described in FIG. 15 comprises a spring that is configured to compress the gas bladder, and wherein the cam is configured to overcome the spring resistance and lift the actuation foot to decompress the gas bladder. Thus, in some cases, the gas bladder is fully decompressed when the actuation foot is lift off the gas bladder, while the gas bladder is fully compressed when actuation foot pushes down on the gas bladder. In some embodiments, when the actuation foot pushes down on the gas bladder, the gas bladder wall is placed under tension. Accordingly, when the actuation foot is lifted up, the gas bladder wall will then retract as well as the gas bladder decompresses.

As described herein, in some embodiments, the eccentric cam of a gas bladder actuation assembly is configured to compress/decompress the gas bladder via a lever arm directly and/or through a spring. For example, the lever and/or spring can be operated in a mode in which the gas bladder is normally compressed, i.e., placed under tension, via the lever and/or spring. The actuation of the cam against the lever or spring causes the lever or spring to retract from the gas bladder decompressing the gas bladder (for example by causing the lever to be lifted or permitting the spring to retract). Alternatively, the gas bladder can be operated in a mode in which the gas bladder is normally decompressed. The actuation of the cam against the lever or spring causes the lever or spring to compress the gas bladder. Thus, in some cases, the lever and/or spring can exert a compression or decompression effect, and the cam the opposite effect (from the lever and/or spring).

In some embodiments, the motor and cam can move through 4 unique rotational positions: Position A) Home position, wherein the actuation foot is fully raised off the gas bladder, and thus the gas bladder is compressed by the spring; Position B) Bladder decompressed fully, wherein the actuation foot is in contact with the gas bladder; Position C) Bladder decompress started, wherein the actuation foot is a few steps up from a fully compressed gas bladder position; and Position D) Away position, wherein the actuation foot is fully compressed under spring force, and where there is no cam contact. In some embodiments, for any gas bladder actuation assembly described herein, the home position, the away position, and the intermediate positions may correspond to the opposite gas bladder position (for example, the home position may correspond to the gas bladder being fully decompressed, and the away position may correspond to the gas bladder being fully compressed).

The home position includes a stop that interferes with the cam rotation so it can trigger a stall detect in the motor controller to know the cam is fully home (e.g., bladder is compressed). The bladder will be fully decompressed (position B) before the cam reaches home. This position corresponds to the number of steps between positions (B) and (C) so that the fluid movement duration can be consistent. Position (C) enables to minimize the variability in the fluid movement time and in the duration of incubation, by moving the cam or foot fully away and then backing off to the point that the strip is just beginning to decompress, thereby signifying the very next step of the motor will move the fluid. This will therefore minimize the delay for the motor to move the eccentric wheel in a position for decompression of the gas bladder, and thereby minimizes the variability in fluid movement across the microfluidic strip.

In some embodiments, the diagnostic reader includes software for storing home and away step counts in a memory, such that one or both of these steps counts can be used to store calibrated values for (B) and (C) for later use in assay testing.

For any gas bladder actuation system embodiment described herein, the diagnostic reader may use a motor controller circuit to drive motor (e.g., 134), that in turn drives a cam (e.g., 136) that interfaces to an actuation foot (e.g., 130), and that foot in turn reduces or increases pressure on the test strip (e.g., 100) gas bladder. As described herein, in some embodiments, a spring applies pressure on the gas bladder (e.g., via the actuation foot), and wherein the motor 134 and cam 136 are configured to reduce such pressure by lifting the actuation foot.

In some embodiments, the motor controller can drive the cam (e.g., 136) through about 330 degrees of rotation, and about 30 degrees of that rotation are able to actively change the gas bladder volume and move fluid. The cam stroke can range from about 0.5 mm to about 3 mm, from about 1 mm to about 2 mm, or from about 1 mm to about 1.5 mm. The compression distance (e.g., the gas bladder air gap) can be about 0.05 mm to about 3 mm, about 0.5 mm to about 2 mm, or about 1 mm to about 1.5 mm. The motor can comprise about 10-30, about 15-25, or about 18-22 total motor steps per rotation (which may include whole steps). The motor gear ratio can be about 96:1. In some embodiments, the motor (e.g., 134) can comprise a stepper motor.

In some embodiments, the motor controller and the motor itself can be operated from about 3.0V to about 10.0V, for example 5.0V. In some embodiments, low (8.5 Ohm) motor coil resistance helps enable detection of motor stall in the motor controller. In some embodiments, the diagnostic reader is configured to sense resistance to the rotation of the motor, wherein said resistance (e.g., force feedback) can be used to detect the limits of travel of the cam.

For example, in some embodiments, this motor controller includes a stall detect feature to know when the motor (e.g., 134) has reached the end of its travel (hit a mechanical “stop”). The stall detect feature can provide feedback that can serve as a proxy to know that the gas bladder is fully depressed. For example, if the position of the cam is not detected correctly and it attempts to rotate beyond the home and bladder down (away) positions, then it could permanently damage the instrument. It is also possible that some damage to the motor may occur if the user attempts to remove the microfluidic strip before the actuation foot of gas bladder actuation assembly is lifted. The stall detect features can detect the phase relationship of the back-EMF (electro motive force) to the motor current. When they are in-phase, the motor is at maximum torque load; and when they are out of phase, the motor is at minimum torque load.

FIG. 16 depicts another exemplary mechanism for compression and/or decompression of a gas bladder as described herein. In some embodiments, the mechanism comprises an actuation foot that presses on the gas bladder, wherein a solenoid may be used to raise and lower the actuation foot for gas bladder decompression and recompression respectively. In some embodiments, a switch is used to trigger a change in position of the actuation foot. In some embodiments, the switch is operatively connected with a timer, such that movement of the actuation foot be can be preconfigured, such as staggered movement of a liquid sample within the microfluidic device for i) allowing the liquid sample to interact and incubate with one or more reagents; ii) moving the liquid sample and reagents to a detection zone for collection by a magnet (as described herein), and/or iii) continued movement of the liquid sample through the microfluidic device to move any particles and the liquid sample not collected in the detection zone (by the magnetic field) away from the detection zone. In some embodiments, the timer may be configured to re-compress the gas bladder, such that instead of moving the uncollected particles and liquid sample in a distal direction, the re-compressed gas bladder moves the liquid sample back to the sample introduction zone (sample application zone). In some embodiments, the timer is configured to compress and decompress the gas bladder in an oscillatory manner, thereby enabling the sample liquid to move back and forth (for example along a portion of the microfluidic device), thereby enabling increased mixing and interaction with the reagent. The timer as described herein can be implemented with any embodiment described herein, including the mechanism disclosed in FIG. 15. In some embodiments, the bladder release button causes the bladder actuation foot (shown with arrow next to spring and damper) to be released as the microfluidic device (e.g., strip) is inserted, thereby enabling the microfluidic device to be inserted under the actuation foot.

For any embodiment described herein, in some cases, an actuation foot is configured (for example, via a cam motor, piezoelectric actuator, or solenoid, as described herein) to move a gas bladder to various positions between a fully decompressed state and fully compressed state. For example, in some embodiments, the gas bladder moves from a fully a compressed position to a fully decompressed position, thereby moving the liquid sample across the microfluidic network, through the reagent zone, the detection zone, and in some cases, through and/or to the gas bladder. In some embodiments, the gas bladder is configured to move from a fully compressed position to a first partially-decompressed position, such that the liquid sample moves from the sample introduction port (and in some cases the capillary stop) to the reagent zone (as described herein for any microfluidic strip) and/or the detection zone. In such cases, for example, the liquid sample can be allowed to incubate with the reagents when the gas bladder is in the first partially-decompressed position for example. The gas bladder can then move from a first partially-decompressed position to a second partially-decompressed position, thereby enabling the liquid sample (complexed with the reagents) to move to the detection zone, for example. The gas bladder can then, in some cases, be moved to a fully decompressed position to move the liquid sample past the detection zone. In some cases, the gas bladder moves from a first partially-decompressed position to a fully decompressed position, wherein the liquid sample moves to the detection zone and/or past the detection zone. In cases where the fully decompressed gas bladder moves the liquid sample to the detection zone, the gas bladder can then be re-compressed to push the liquid sample out of the detection zone and back across the microfluidic device, optionally expelling from the sample introduction port.

In some embodiments, for any embodiment described herein, the movement of the liquid sample through the microfluidic device is facilitated via the open channel of the microfluidic network on the microfluidic device. For example, the lack or minimal use of a porous pathway reduces interference for fluid flow and/or allows for accurate fluid flow control. In some cases, a porous media may be used within the microfluidic network.

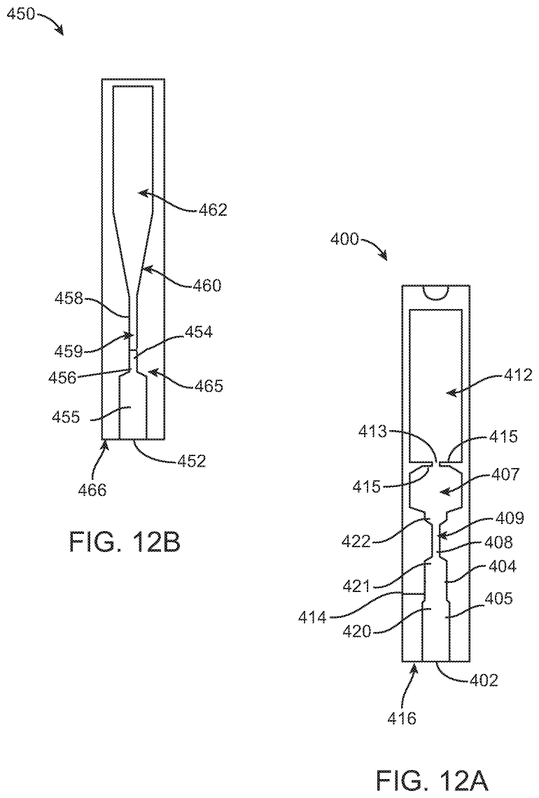

FIG. 12b is a top view of a second embodiment of an end fill diagnostic strip 450, which may be operated by a diagnostic reader (instrument) such as the diagnostic reader shown in FIGS. 2 and 3, as discussed for the end fill strip 100 of FIG. 1, and wherein the liquid sample can be first metered in the liquid sample metered portion 455, before moving across the reagent zone 454. The end fill strip 450 is formed of materials and layers as discussed for the end fill strip 100 of FIG. 1. Proceeding from a proximal periphery 466 of the end fill strip 450, the end fill strip 450 has a microfluidic network including a sample application zone 452 (sample introduction port), reagent zone 454, a liquid sample metered portion 455, a capillary stop 456, a detection channel 459 including a detection zone 458, a transition zone 460, and a gas bladder 462. The end fill strip 450 also includes a vent channel (not shown on FIG. 12b) having a first opening disposed within the microfluidic network at the capillary stop and a second opening disposed adjacent a lateral periphery of the strip as described for the end fill strip 100 of FIG. 1. In some cases the adhesive layer 465 of the end fill strip 450 of FIG. 12b is opaque (black) rather than translucent. The liquid sample metered portion 455 allows for liquid sample to collect on the microfluidic device up to the capillary stop, thereby providing a metered, predetermined volume of the liquid sample to be assayed.

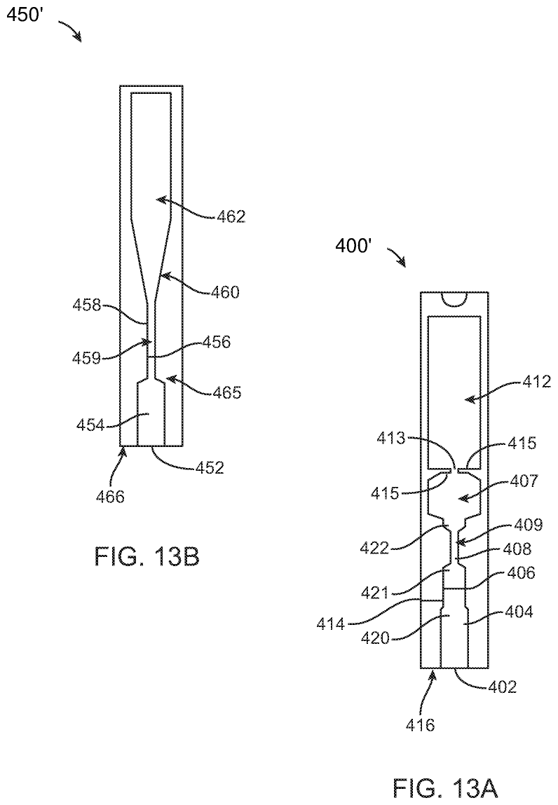

Except for the gas bladder 462 and transition zone 460, the dimensions of the end fill strip 450 of FIGS. 12b, 13b can the same as for the end fill strip 100 of FIG. 1, and/or as described further herein. For example, the length of the transition zone 460 of the end fill strip 450 of FIGS. 12b, 13b is from about 9 mm to about 13 mm, such as 11 mm. The width of the transition zone 460 of the end fill strip 450 of FIGS. 12b, 13b increases from the 0.65 mm approximate width of the detection zone 308 to the 4.8 mm full approximate width of the gas bladder 462 by about 0.5 mm per mm traversed along the length of the microfluidic network (e.g., increasing with an angle of about 27 degrees). The gentler transition of the transition zone 460 of the end fill strip 450 of FIGS. 12b, 13b as compared to the transition zone 110 of the end fill strip 100 of FIG. 1, reduces the likelihood that portions of the sample liquid will separate from one another (which could introduce interfering bubbles) when the sample liquid is drawn through the detection 459 channel and into the gas bladder 462 during operation of the end fill strip 450.

FIG. 13b depicts a top view of another version (450′) of the end fill strip of FIG. 13a, wherein the capillary stop 456 is located distal to the reagent zone 454. In such embodiments, the liquid sample may fill through the reagent zone to the capillary stop when applied to the sample application zone (sample introduction port) 452.

FIG. 12a is a top view of another embodiment of an end fill diagnostic strip 400, which may be operated by a diagnostic reader (instrument) such as the diagnostic reader 200 shown in FIGS. 2 and 3, as discussed for the end fill strip 100, 450 of FIGS. 1 and 13. The end fill strip 400 is formed of materials and layers as discussed for the end fill strip 100, 450 of FIGS. 1 and 13. Proceeding from a proximal periphery 416 of the end fill strip 400, the end fill strip 400 has a microfluidic network including a sample application zone (sample introduction port) 402, reagent zone 404, a capillary stop in fluid communication with a vent 414, a liquid sample metered portion 405, a detection channel 409 including a detection zone 408, an overflow reservoir 407, and a gas bladder 412 spaced apart from the overflow reservoir by a pair of reservoir supports. The overflow reservoir 407 and gas bladder 412 are in gaseous communication via a bladder opening 413 therebetween. The end fill strip 400 of FIG. 12a also includes a vent channel 414 having a first opening disposed within the microfluidic network at the capillary stop (not shown) and a second opening disposed adjacent a lateral periphery of the strip as described for the end fill strips 100, 450, 450′ of FIGS. 1 and 12b, 13b. The dimensions of the end fill strip 400, 400′ of FIGS. 12a, 13a can be similar to those disclosed for the end fill strips of FIGS. 1 and 12b, 13b, and/or as further described herein.

In use, the end fill strip of FIG. 12a is operated as disclosed for the end fill strips 100, 450 of FIGS. 1 and 12b. Prior to the application of sample liquid to the application zone 402 (sample introduction port), the gas bladder 412 is compressed. Sample liquid (liquid sample) flows by capillary action through the microchannel until a distal liquid-gas interface of the sample liquid reaches the capillary stop (in fluid communication with vent 414) at which point capillary flow ceases. Typically, the capillary stop is located proximal to, i.e., upstream of, the reagent zone. The sample liquid may be detected at this location via sensor location at 420 (as described herein). Subsequently, the gas bladder 412 is decompressed until the distal liquid-gas interface of the sample liquid has moved through the reagent zone to a location distal to, i.e. downstream of, the reagent zone. After sensing the presence of the liquid sample at this distal location (as described herein, for example via sensor location at 421), the bladder decompression is ceased with the liquid sample in contact with the mobilizable reagents in the reagent zone. After an incubation period in which the sample liquid mobilizes the reagents within the reagent zone 404, the gas bladder 412 is decompressed at a controlled rate to draw the sample through the detection zone 408 and into the overflow (overfill) reservoir 407, e.g., at least until the proximal gas-liquid interface of the liquid sample has moved through the detection zone to a location distal the detection zone (which may be detected by a sensor at location 422, as described herein). As the sample liquid passes through the detection zone 408, the magnetic reagent (which may have been previously located in the reagent zone) is retained in the detection zone as disclosed for the end fill strip of FIG. 1. When the gas bladder 412 of the end fill strip of FIG. 12 is in the compressed state, the reservoir supports 415 limit or prevent compression of the overflow reservoir. In a compressed state, a height of the overflow reservoir between internal lower and upper surfaces thereof would decrease. The decrease in height of the overflow reservoir 407 would increase the capillary force experienced by sample liquid therein. The increased capillary force can separate portions of the sample liquid entering the overflow reservoir 407 from more proximal portions still located within the detection zone thereby reducing the precision by which the position and movement of the sample liquid can be controlled by compression and decompression of the gas bladder 412. Accordingly, the reservoir supports 415 permit the use of a compressible gas bladder 412 in gaseous communication with an adjacent overflow reservoir 407 in a compact format without compromising the precise control of sample liquid position and movement.

FIG. 13a depicts a top view of another version (400′) of the end fill strip of FIG. 12a, wherein the capillary stop 406 is located distal to the reagent zone 404. In such embodiments, the liquid sample may fill through the reagent zone to the capillary stop when applied to the sample application zone (sample introduction port) 402.

As described herein, for any microfluidic device described herein, such as any end fill strip (e.g., FIGS. 12a-b, 13a-b) or top fill strip, the device (e.g., strip) may have a length from about 40 mm to about 50 mm, such as about 45 mm, and a width from about 6 mm to about 10 mm, such as about 8.5 mm. In some embodiments, the microfluidic network, as disposed on the strip, may have a width of about 5 mm to about 8 mm, such as about 6.5 mm. In some embodiments, the sample introduction port (and optionally a portion of a channel directly distal to the sample introduction port on the microfluidic device, such as the liquid sample metered portion described herein) has a width from about 2 mm to about 5 mm, such as about 3.2 mm. In some embodiments, a portion of the reagent zone (e.g. 404 of FIG. 12a), and/or a portion in microfluidic network where the capillary stop is located (e.g., 406 of FIG. 13a), may have a width of about 2 mm to about 3 mm, such as about 2.5 mm. In some embodiments, the detection channel, including the detection zone, may have a width of about 0.5 mm to about 1.5 mm, such as about 0.89 mm. In some embodiments, for any channel within the microfluidic network, the channel may have a depth of about 0.05 mm to about 0.2 mm, such as about 0.11 mm. As described herein, in some cases, the channel depth is defined by the respective adhesive located on the top and bottom substrate of the microfluidic device.

In some embodiments, the length of reagent zone and channel to the capillary stop is from about 5 mm to about 15 mm, such as about 8 mm, about 10 mm, or about 12 mm. In some embodiments, the length of the detection zone is about 3 mm to about 6 mm, such as about 4 mm. In some embodiments, the length of the overfill reservoir is from about 4 mm to about 10 mm, such as about 6 mm. In some embodiments, the length of the gas bladder is from about 15 mm to about 25 mm, such as about 18 mm. In some embodiments, the reservoir supports (e.g., see FIG. 12, reference character 415) have a thickness from about 0.25 mm to about 3 mm, such as about 0.5 mm or about 1.5 mm.

FIGS. 5a-9b depict an exemplary microfluidic device that is a top fill strip 300, and further depicts various components of a diagnostic reader as aligned in relation with the top fill strip when the top fill strip is inserted within the diagnostic reader. For example, FIG. 5a depicts a top view of a top fill strip, having a sample introduction port 302, a reagent zone 303, a vent 304, a capillary stop 306, a detection zone 307, and a gas bladder 316. In some embodiments, the capillary stop 306 is located proximal (i.e., upstream) of the reagent zone 303 (i.e. the capillary stop is closer to the sample introduction port 302 than the reagent zone 304).

The strip 300 is depicted as having a length of about 50 mm and a width of about 10 mm. Moreover, the gas bladder 316 is depicted as having a volume of about 40 μl. FIG. 5b depicts a side view of the strip 300, and further depicts components of a diagnostic reader, such as a piezoelectric actuator and optionally actuation foot 310, a magnet 309, a magnet shield 308, an optical detection assembly (comprising for example an LED and photodiode) 312, and a gas bladder actuation foot 314. FIG. 5b depicts an exemplary alignment between the components of the diagnostic reader the strip 300 when inserted within diagnostic reader. The depicted alignment may be applicable for any microfluidic device described herein, such as the end fill strip. As described herein, in some cases, the end fill strip comprises a similar configuration to that of the top fill strip, with at least one difference being the location of the sample introduction port, which is located at a periphery of a proximal side of the end fill strip. In some embodiments, the strip 300 further comprises an upper substrate 317 (optionally having an adhesive layer, as described herein), and a lower substrate 318 (optionally having an adhesive layer, as described herein). For any embodiment of a microfluidic device, the microfluidic device may have one or more channels for liquid sample to flow therethrough. For any embodiment of a microfluidic device described herein, the gas bladder 316 can have a depth that is greater than a depth of any channel on the microfluidic network of the microfluidic device. In some embodiments, the magnet 309 and magnet shield 308 can be disposed above the microfluidic device (shown as below in FIG. 5b), and/or the optical detection assembly 312 can be disposed below the microfluidic device (shown as above in FIG. 5b).

FIG. 5c depicts a side view of the strip 300, wherein a volume of liquid sample (e.g., 20 μl) is applied to the sample introduction port 302.

FIG. 6a depicts the strip 300 of FIG. 5a. FIG. 6b depicts a side view of the strip 300 (of FIG. 5a), wherein a gas bladder actuation foot 314 pushes down on the gas bladder 316, thereby compressing the gas bladder. In some embodiments, a button pushed on the diagnostic reader, after a strip described herein is inserted therein (e.g., in an operatively secure state), enables the actuation foot to push down on the gas bladder (as described herein).

FIG. 7a depicts a top view of the strip 300, wherein a liquid sample has been applied through the sample introduction port, and wherein the liquid sample flows via capillary action to the capillary stop. FIG. 7b depicts a side view of the strip 300 with the liquid sample in FIG. 7a.

FIG. 8a depicts a top view of the strip 300 from FIG. 8b, which depicts an optional step of enhancing mixing and interaction between the liquid sample and the reagents within the reagent zone. For example, in some case, a piezo actuator, optionally along with an actuation foot 310 are configured to oscillate against the upper substrate, thereby causing the channel within the microfluidic network of the strip 300 to compress and decompress (in some case a subtle compression/decompression). In some cases, such oscillation also results or instead results in vibration. In some cases, such compression/decompression and/or vibration promotes interaction and mixing of the liquid sample with the reagent. In some cases, the piezo actuator enables for a range of frequencies to be applied across the liquid sample to enhance mixing and/or interaction between the liquid sample and the reagents.

As described herein, in some embodiments, the capillary stop 306 is located proximal to the reagent zone 304. Accordingly, the liquid sample will first flow to the capillary stop, wherein a metered volume of the liquid sample may be collected. In some cases, the liquid sample is then moved to the reagent zone (e.g., via partial decompression of the gas bladder), so as to enable mixing and incubation of the liquid sample with the reagents, as described herein.

FIG. 9a depicts a top view of the strip 300, wherein the liquid sample has moved through the detection channel and into the gas bladder 316. FIG. 9b depicts a side view of the strip 300 of FIG. 9a, wherein the liquid sample is shown to have moved from the reagent zone through the detection zone (and thus past the magnet 309) to the gas bladder 316, as a result of the actuation foot being lifted off the gas bladder. In some embodiments, the lifting of the actuation foot off the gas bladder can be in a controlled manner, and occur over a given time period (e.g., about 2 minutes, about 3 minutes, about 4 minutes, about 5 minutes, about 6 minutes, about 7 minutes, about 10 minutes). In some embodiments, such controlled decompression of the gas bladder enables the liquid sample to move through the microfluidic network in a controlled manner, thereby enabling the magnetic field exerted across by the magnet to collect magnetic reagent particles in the detection zone, which may be complexed with a target within the liquid sample. Accordingly, the collected magnetic reagent particles may be exposed to a light (via an LED from the optical detection assembly), wherein a signal may be emitted by the particle (e.g., fluorescence signal) that can be detected by a photodiode (on the optical detection assembly), and thereby correlating to the presence and/or amount of a target in a sample.

FIGS. 2, 3, and 11a-11e depict an exemplary diagnostic reader as described herein. As shown in FIGS. 2 and 3, the diagnostic reader 200 includes a microfluidic device introduction port 202 for receiving a microfluidic device as described herein. The diagnostic reader may further comprise buttons for enabling the microfluidic device to be inserted in an operatively secure state within the diagnostic reader. The buttons may also enable actuation of one or more mechanisms within the microfluidic device, such as for compressing/decompressing the gas bladder (as described herein), operating a light source to enable detection of a detectable label (e.g., optical label) with the liquid sample and reagent mixture, and/or actuate one or more mechanisms to help enable further mixing and interaction between the liquid sample and the reagents (as described herein).

FIG. 11a depicts the diagnostic reader of FIG. 2. FIGS. 11b-11e depict exemplary components within the diagnostic reader, such as a notch 315 to align the microfluidic device within the diagnostic reader (FIG. 11b). FIG. 11c depicts an exemplary actuation mechanism for further mixing the liquid sample and reagent, the mechanism comprising a spring and transducer. FIGS. 11c-11d depict an exemplary fixed magnet along with an exemplary shield (spacer) that is aligned with an optical detector (such as a photodiode), for detection of a signal (e.g., fluorescence) emitted by a detectable label of a reagent that is irradiated with a light (e.g., in the detection zone of a microfluidic device). FIG. 11e depicts an exemplary orientation of a magnet 122 and spacer 124, fixed in a position within the diagnostic reader, and wherein the length of the magnet and spacer extend across a width of the diagnostic reader (and therefore microfluidic device when inserted therein). FIG. 1 if depicts an exemplary microfluidic device, as described herein, which is inserted within the diagnostic reader.

In some embodiments, the fixed magnet and an optical detection system are aligned within the detection reader. As described herein, one or more targets within a liquid sample may be detected optically, based on a detection label (e.g., optical detection label) of a reagent that is configured to bind to a target, and that is also collected in the detection zone of a microfluidic device (via the magnetic field generated from the fixed magnet, as described herein). The optical detection system may comprise a light source for irradiating the detection zone of a microfluidic device (and therefore detection labels within the detection zone), and an optical detector for detecting a signal emitting from the detectable labels collected in the detection zone. In some cases, the signal is fluorescence emitted by the detection labels upon irradiation.

In some embodiments, the diagnostic reader is configured to detect a liquid sample and corresponding location within a microfluidic device (located within a diagnostic reader). In some embodiments, such detection of the liquid sample is performed optically. For example, in some cases, the diagnostic reader comprises one or more optical sensors (such as reflective optical sensors) that can be used to detect i) the presence and/or absence of a microfluidic device within a diagnostic reader, ii) that the microfluidic device is inserted fully within the diagnostic reader (e.g., operatively secure state), and/or iii) detect liquid sample movement across the microfluidic device. Such detection by the optical sensors may be via measuring transmittance of light passing once through the microfluidic network (e.g., a channel) or by measuring the transmittance of light passing twice through the channel (being reflected after the first pass). The presence of the liquid sample fluid front can be determined by the change in transmittance resulting from the gas-liquid interface passing through the ‘detection’ zone. In some embodiments, the diagnostic reader comprises optical sensors disposed at different locations for detection of the liquid sample at such different locations (on the microfluidic device, and within the diagnostic reader).

In some embodiments, the presence of a microfluidic device (e.g., end fill strip, top fill strip, etc.) upon insertion to the diagnostic reader is detected at the start of the test to initiate the test process. During the test process, the reader can detect if the strip is removed and the software (associated with the diagnostic reader) can generate an error. At the end of the test process, to the reader can confirm that the strip has been removed as instructed.

As described herein, two more optical sensors of this type are used at a second position on the strip to detect that the sample is applied to the test strip and that the fluid hasn't travelled too far. In some cases, this is critical because it is used to start the incubation timer—for example, the amount of time required for the sample and the reagent to mix completely. In some cases, another optical sensor of this type is used at another position on the strip to detect that the sample has moved into the wash reservoir, and thus confirming proper fluid movement

FIGS. 12a, 13a depict exemplary locations (420, 421, and 422) for one or more optical sensors (as described herein). In some embodiments, such optical sensors enable detection of a sample liquid within the microfluidic network. For example, in some embodiments, the strip channel design and optical sensor positions are matched to enable detection of sufficient liquid sample applied, wash detection, as well as enabling other fluid movement control functions (for example via the diagnostic reader) and detection of correct fluidic functioning of the assay. In some embodiments, the sensors can be used to detect dry to wet and/or wet to dry transitions.

Accordingly, in some embodiments, the diagnostic reader is configured to control the fluid position, and/or confirm the fluid front meniscus has passed a particular sensor and/or the trailing edge meniscus has passed a particular sensor during the running of the test. In some embodiments, an optical sensor is located within the liquid sample metering portion 405 and/or close to the capillary stop (for example, see 420 in FIG. 12a), to detect the presence of the liquid sample on the strip. In some embodiments, an optical sensor is located at or close to the entry of the detection channel, which may be within or distal to the reagent zone (for example, see 421 in FIG. 12a), so as to detect movement of the liquid sample to the reagent zone. In some embodiments, an optical sensor is located within the overfill reservoir region (for example, see 422 in FIG. 12), so as to detect movement of the liquid sample beyond the detection channel and detection zone. For example, in some embodiments, if the liquid sample reaches and subsequently clears the sensor 422 according to expected assay timings, then the meniscus wash is complete and is clear of the detection zone.

Described herein is an exemplary method of operation for a diagnostic reader described herein used to detect the presence and/or amount of a target in a liquid sample. First, a door of the diagnostic reader (e.g., see diagnostic reader 200) is opened and powered up. The diagnostic reader may then prompt for a microfluidic device (e.g., top fill strip as described herein, end fill strip as described herein) to be inserted within the diagnostic reader, wherein the diagnostic reader is configured to detect the insertion of the microfluidic device therein (for example, via optical sensors as described herein). Once the microfluidic device has been inserted within the diagnostic reader, the diagnostic reader may then prompt for the sample (e.g., liquid sample as described herein) to be applied to the microfluidic device. In some cases, the applied sample is detected by the diagnostic reader (for example, via optical sensors as described herein). Upon detection of the sample (or application of the sample) on the microfluidic device, the diagnostic reader may prompt for a door to be closed on the reader for the assay to be performed. The diagnostic reader then begins the detection process, where the liquid sample may be incubated with the reagents, then the liquid sample then moves through the microfluidic device so as to enable detection for the presence and/or amount of a target in the liquid sample (via the optical detection system as described herein). The diagnostic reader calculates a result (of the detection), and in some cases, a positive or negative result is displayed. In some embodiments, the results continues to display while the diagnostic reader prompts for the microfluidic strip to be removed from the reader. The diagnostic reader will power down after a short timeout after the microfluidic device has been removed, and the door to the reader is closed.

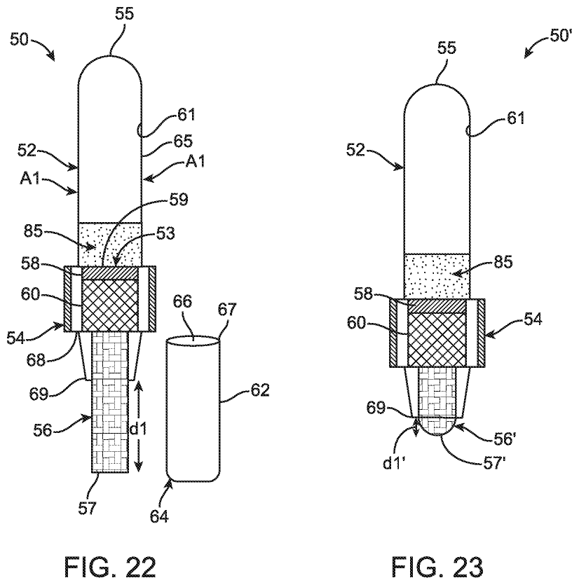

FIGS. 22-26b depict various exemplary embodiments of applicators for applying a liquid sample to any microfluidic device described herein (e.g., end fill strip, top fill strip).

Referring to attached FIG. 22, an extraction vial 50 is configured for preparing a liquid mixture 85 composed of a liquid buffer and a biological specimen to facilitate the determination of the presence and/or amount of one or more targets present in the biological specimen. Extraction vial 50 is further configured for applying liquid mixture 85 directly to a sample introduction port (e.g., sample application zone) of a microfluidic device. For example, extraction vial 50 may be used to apply liquid mixture 85 to any of the microfluidic devices (e.g., strips) disclosed herein such as the microfluidic strips of FIGS. 1, 5A-9B, 17 or 18, or any of the strips disclosed in the '325 Application. The microfluidic device may include one or more microchannels defined by non-porous, impermeable substrate(s) with microchannels that are open, e.g., not filled or occupied by, e.g., is free of, a solid phase or porous membrane through which liquid must pass. Such microfluidic strips may be used with any compatible diagnostic reader including any of the diagnostic readers disclosed herein such as, e.g., the diagnostic reader 200 of attached FIG. 2 or 3 or any of the readers disclosed in the '325 Application.

Extraction vial 50 includes a tubular body 52 having a base 55, a body opening 53, an applicator cap 54, and a sealing cap 64. The tubular body can be removably coupled with the applicator cap. Applicator cap 54 includes a two-stage filter having a glass filter layer 58 and a foam filter layer 60. Foam filter layer 60 may be formed of a polymer such as polyurethane or polyethylene. In embodiments, the foam filter layer is sintered, e.g., of sintered beads such as beads having a diameter between about 5 microns and 15 microns or about 10 microns. At least some of the beads, e.g., between about 15% and 50%, e.g., about 30%, of the total beads of the filter may be ion exchange beads of similar size to the remaining beads.

Applicator cap 54 further includes a porous applicator 56. Sealing cap 64 includes a generally tubular body 62 with a cap opening 66. Sealing cap 64 is configured to accommodate porous applicator 56 therein such that a lip 67 of cap opening 66 engages a distal shoulder 68 of applicator cap 54 via a snap-fit or other securing mechanism, e.g., threads, to prevent liquid mixture from exiting extraction vial 50. Applicator cap 54 engages tubular body 52 via threads (not shown) or other securing mechanism (e.g., a snap-fit).

Applicator 56 includes a proximal tip 57 by which liquid mixture 85 held by tip 57 is applied to a sample application zone of a microfluidic strip (described herein) as discussed below. Tip 57 extends a distance d1 beyond a proximal shoulder 69 of applicator cap 54. Distance d1 is typically at least about 2 mm, e.g., at least about 3 mm and may be about 15 mm or less, about 10 mm or less, or about 7.5 mm or less. The proximal extension of tip 57 beyond proximal shoulder 69 of applicator cap 54 facilitates the application of liquid held by tip 57 to a sample application zone of a microfluidic device (as described herein) without interference by proximal shoulder 69 applicator cap 54. At least a portion of applicator 56 includes a sufficiency indicator that responds to the presence of liquid therein. The sufficiency indicator may be localized within tip 57 or may be distributed through substantially all, e.g., all of applicator 56. The sufficiency indicator changes color or other optical characteristic when sufficient sample liquid is present within applicator 56. During use, the sufficiency indictor alerts a user that liquid has reached tip 57 and is ready to apply to a microfluidic device.

Extraction vial 50 may be provided to a user with an amount of liquid buffer stored within tubular body 52 and a with a removable seal (not shown) covering body opening 53. The removable seal is typically composed of a metallic foil, a polymer layer, e.g., a polymer film, or combination thereof to prevent the passage of the liquid buffer through body opening 53 as well as preventing the evaporation of the liquid buffer therein. An exemplary seal is a multilayer seal having a thickness of about 0.025 mm and formed of polymer-metal-polymer layers, e.g., polyethylene-aluminum-polyethylene. The seal may be heat staked to the tubular body.

Tubular body 52 is formed of polyethylene or other polymer and an internal surface 61 thereof may be treated to reduce adsorption or non-specific binding of proteins or other targets to be determined in liquid mixture 85. Tubular body 52 is deformable so that an outer surface 65 thereof can be compressed as indicated by arrows A1 to increase pressure therein and expel the liquid mixture through filters 58,60 and porous applicator 56 as discussed below. As illustrated in attached FIG. 22, extraction vial 50 is in an application orientation with liquid mixture 85 in contact with an exposed surface 59 of glass filter 58.

The composition of the liquid buffer depends on the type of biological specimen and target(s) for which the extraction vial is to be used. For example, in some embodiments, the liquid buffer is configured to lyse cells present in the biological specimen, whereas in other embodiments the liquid buffer is configured to preserve cells intact, without lysing or otherwise releasing the contents thereof. The liquid buffer may include a blocking agent, e.g., a protein-based blocking agent, e.g., a protein-based blocking agent such as bovine serum albumin to reduce non-specific binding of proteins or other biological compounds to inner surface 61 of tubular body 52 and/or to components of filters 58,60 or porous applicator 56.

Extraction vial 50 can be operated as follows in a workflow for determining the presence and/or amount of one or more target(s) present in a biological specimen. Exemplary biological specimens may be collected from a mammal, e.g., a human being, and include a nasal, salivary, throat, nasopharyngeal, mid turbinate, urine, or vaginal specimen. Biological specimens may be collected with a collection swab or other suitable specimen collection tool. Once the biological specimen has been obtained, a user positions extraction vial 50 in a non-application orientation in which the liquid buffer gathers adjacent base 55 and opposite opening 53 of tubular body 52. The user removes the removable seal to expose body opening 53 and the liquid buffer therein. The user inserts the collection tool carrying the biological specimen, e.g., a tip of a collection swab, into the liquid buffer and agitates the tool to combine the biological specimen and liquid buffer, forming a liquid mixture to be subjected to analysis. The user may compress outer surface 65 of tubular body 52 as shown by arrows A1 to squeeze the collection tool releasing more biological specimen into the liquid buffer. Once the biological specimen has been released from the collection tool into the liquid buffer, the user may remove the collection tool from tubular body 52. Alternatively, the user may leave the collection tool or a portion thereof within tubular body 52, e.g., by breaking a frangible connection to separate the tip of a collection swab from the remainder of the shaft of the collection swab.

The user then secures applicator cap 54 over body opening 53 so that liquid mixture 85 must pass through filters 58,60 and porous applicator 56 when exiting the tubular body 52. When the user is ready to perform a determination for a target(s) present in liquid mixture 85, the user prepares a microfluidic device for operation by a diagnostic reader, e.g., as disclosed herein for the end fill strip 100, 300, 400 of FIGS. 1, 17, and 18 respectively, or in the '325 Application. The user positions extraction vial 50 in the application orientation such that liquid mixture 85 contacts exposed surface 59 of glass filter 58 and then begins to pass into and through filters 58,60 and applicator 56. Filters 58,60 retain particulates that might otherwise interfere with the determination of a target(s) therein. Liquid (filtrate) of liquid mixture 85 may pass through the filters and applicator via capillary action. Alternatively, or in combination, a user may increase gas pressure within tubular body 52 by squeezing external surfaces 65 along arrows A1 to force liquid through the filters and into applicator 56. When liquid (filtrate) of liquid mixture 85 has reached proximal tip 57 of applicator 56, the sufficiency indicator alerts the user that sufficient liquid (filtrate) has reached tip 57. The tip 57 holds the liquid so that the liquid does not drip unintentionally. The user then contacts the sample application zone of the microfluidic strip with liquid (filtrate) held by tip 57 whereupon liquid enters the microfluidic network of the microfluidic strip as described for sample application to the microfluidic strip of FIGS. 1, 17 and 18. During the application, applicator 56 including applicator tip 57 remains substantially uncompressed, e.g., the volume of applicator 56 or tip 57 is not substantially compressed to reduce the volume thereof and “squeeze” liquid sample from tip 57 or to force liquid into the microfluidic network via compression of the applicator 56. Instead, liquid applied to the sample application port (sample application zone) is drawn by capillary action from the tip 57 of the applicator 56 through the sample introduction and into the microfluidic network of the strip (as described herein). For example, the capillary force within the microfluidic network of the strip may be greater than the capillary force within the applicator tip, thereby enable the liquid sample to flow onto to the microfluidic device.

Once sufficient liquid (filtrate) of liquid mixture 85 has been applied to the sample application zone, the user repositions extraction vial 50 in the non-application orientation and encloses applicator 56 with sealing cap 64 to seal the extraction vial. The diagnostic reader then operates the microfluidic device to determine one or more targets in the applied liquid, e.g., as disclosed herein for the strip of FIGS. 1, 5A-9B, 17, 18, or for any of the microfluidic devices of the '325 Application.

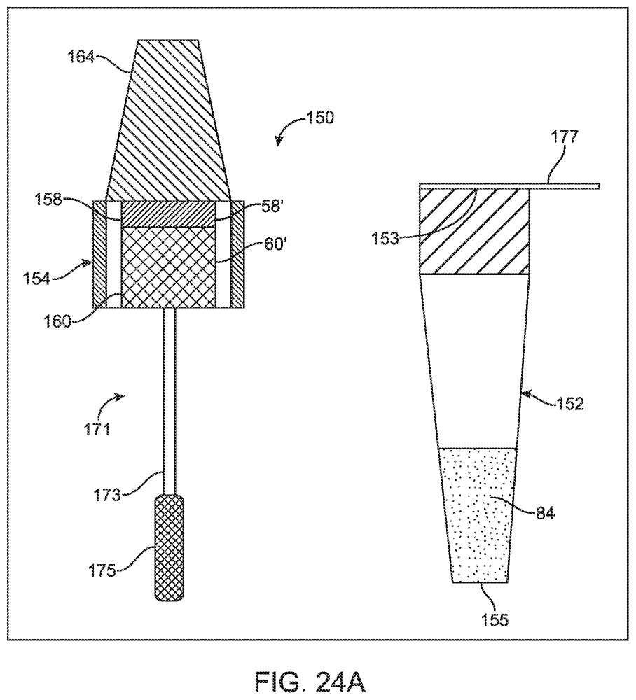

Referring now to attached FIG. 23, an extraction vial 50′ includes a tubular body 52, applicator cap 54, filters 58,60, and sealing cap (not shown) as disclosed for extraction vial 50. Extraction vial 50′ includes a porous applicator 56′ having a rounded proximal tip 57′ that extends a distance d1′ beyond proximal shoulder 69 of applicator cap 54. Distance d1′ is less than distance d1 of tip 57 of applicator 56 of extraction vial 50 (FIG. 22). For example, the maximum extent of distance d1′ may be at least about 2 mm, e.g., at least about 3 mm and may be about 7 mm or less, about 6 mm or less, or about 5 mm or less. Tip 57′ includes a sufficiency indicator as discussed for tip 57 or applicator 56. In use, extraction vial 50′ is operated to prepare a liquid mixture 85 of a buffer liquid and biological specimen and to apply liquid (filtrate) of liquid mixture 85 to a sample application zone of a microfluidic strip as disclosed for extraction vial 50. The behavior of applicator 56′ during application (e.g., with respect to volume and capillary action) is the same as disclosed for applicator 56 of extraction vial 50.

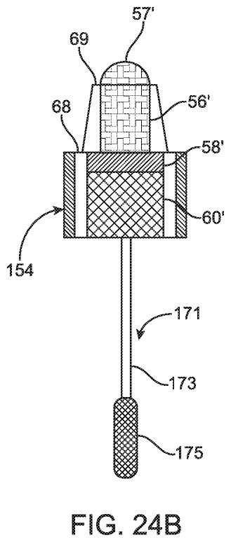

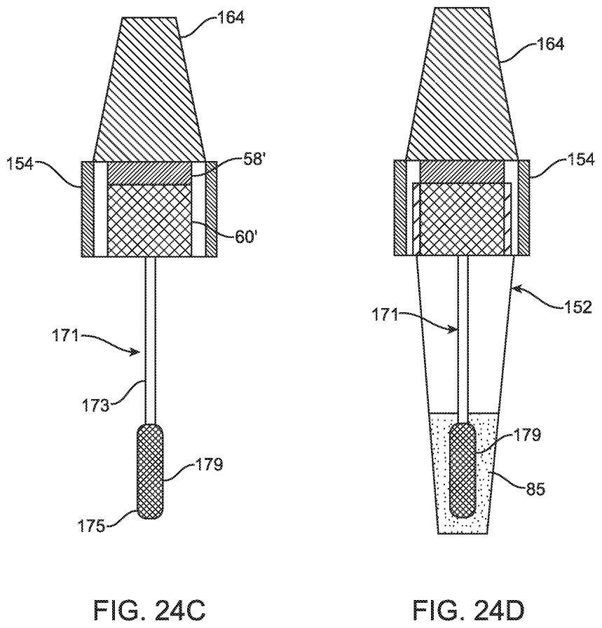

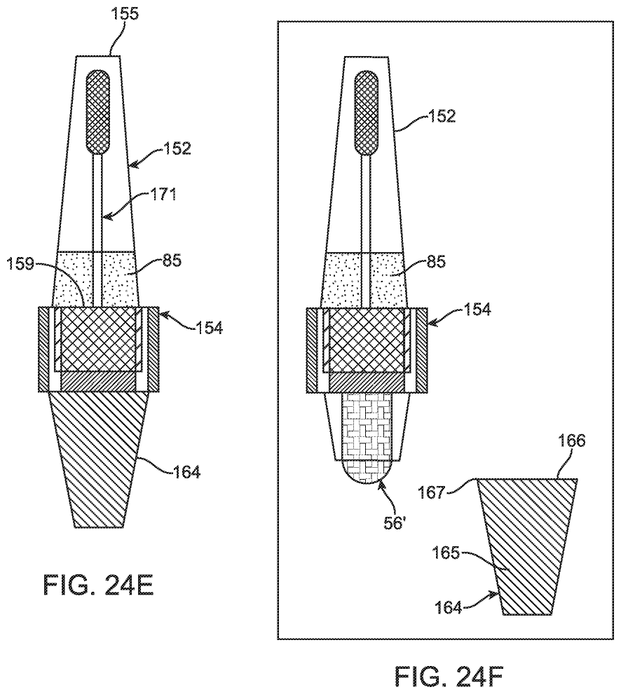

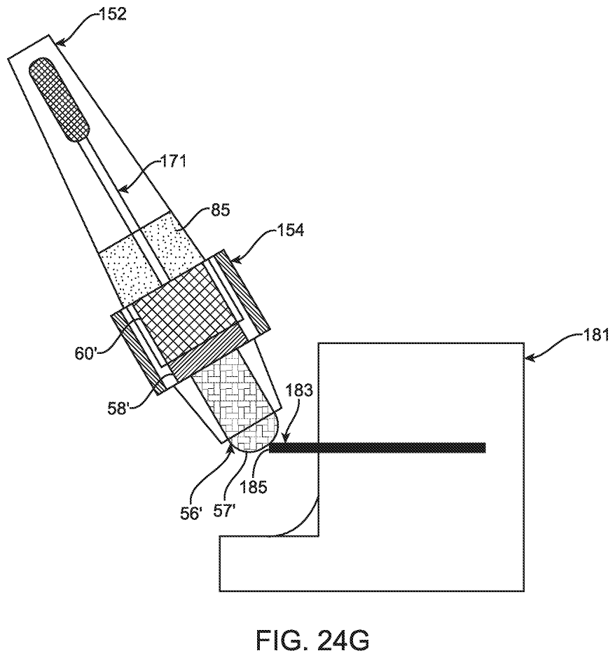

With reference to attached FIGS. 24a-24g, an extraction vial 150 includes a tubular body 152, an applicator cap 154, and a sealing cap 164. As further discussed below, applicator cap 154 includes an integral collection swab 171 having a shaft 173 and a collection swab tip 175. Like extraction vials 50 and 50′, extraction vial 150 is configured for preparing a liquid mixture 85 including a liquid buffer 84 and a biological specimen 179 (see FIGS. 24c and 24d) to permit the determination of the presence and/or amount of one or more targets present in the biological specimen and for applying liquid (filtrate) of liquid mixture 85 directly to a sample introduction port (sample application zone) of a microfluidic device described herein.

In addition to applicator cap 154, extraction vial 150 includes a tubular body 152 having a base 155, a body opening 153, and a sealing cap 164. Applicator cap 154 includes a two-stage filter having a glass filter layer 58′ and a polyurethane foam filter layer 60′. Relative to filters 58,60 of extraction vials 50,50′ (FIGS. 22 and 23), the order of filters 58′,60′ is flipped in extraction vial 150 so that a surface 159 of filter layer 60 of application cap 154 is disposed to directly contact liquid mixture 85 within tubular body 152 (FIG. 24e). Applicator cap 154 also includes a porous applicator 56′. Sealing cap 164 includes a generally conical body 165 with a cap opening 166 (FIG. 24f). Sealing cap 164 is configured to accommodate porous applicator 56′ therein such that a lip 167 of cap opening 166 engages distal shoulder 68 of applicator cap 154 via a snap-fit or other securing mechanism, e.g., threads, to prevent liquid mixture from exiting extraction vial 150. Applicator cap 154 engages tubular body 152 via threads (not shown) or other securing mechanism (e.g., a snap-fit).

Collection swab tip 175 is configured to collect a biological specimen 179, e.g., from a mammal such as a human. Exemplary biological specimens include a nasal, salivary, throat, nasopharyngeal, mid turbinate, urine, or vaginal specimen. Collection swab tip 175 is integrally formed with shaft 173 of sintered or three-dimensionally printed polymer (e.g., polyethylene or polyurethane) although alternative collection swab tips may be used including flocked or spun fiber swab tips, sponge or foam tips, or combinations thereof. Such a sintered swab tip may be formed of different size beads (e.g., different sized polyethylene or polyurethane beads) to give the tip a variable texture.

Tubular body 152 has properties similar to tubular body 52, including material composition, deformability, and reduction of adsorption or non-specific binding. As provided to a user, tubular body 152 includes an amount of a liquid buffer 84 and body opening 153 is sealed with a seal 177 (FIG. 24a). The liquid buffer and seal 177 may have the same properties and composition as the liquid buffer and seal disclosed for extraction vial 50.

The operation of extraction vial 150 is discussed with respect to FIGS. 24c-24g. In FIG. 24c, applicator cap 154 has sealing cap 164 secured thereto and collection swab 171 is ready for use in collecting a biological specimen. For example, a user may use collection swab tip 175 to collect a nasal or nasopharyngeal specimen or another biological specimen as disclosed herein.

In FIG. 24d, an amount of collected biological specimen 179 is held by collection swab tip 175. The user removes seal 177, inserts collection swab tip 175 through body opening 153 into the liquid buffer and secures application cap 154 to tubular body 152. The user then forms liquid mixture 85 of the liquid buffer 84 and collected biological specimen 179 as disclosed for extraction vial 50. Once liquid mixture 85 is formed, the user positions extraction vial 150 as seen in FIG. 24g in an application orientation with liquid mixture 85 in contact with exposed surface 159 of filter 60′. After removing sealing cap 160, the user applies an amount of liquid mixture 85 to a sample application zone 185 of a microfluidic device 183 as disclosed for extraction vial 50 (FIG. 24g). The microfluidic device 183 can be any microfluidic strip described herein (e.g., 100, 300, 400, etc.) The process for applying filtrate of liquid mixture 85 to application zone 185 and the properties of the applicator 56′ during application are the same as for extraction vial 50,50′. As can be seen by comparing FIGS. 24f and 24g, a sufficiency indicator within applicator 56′ has changed color (FIG. 24g) indicating the presence of liquid therein.

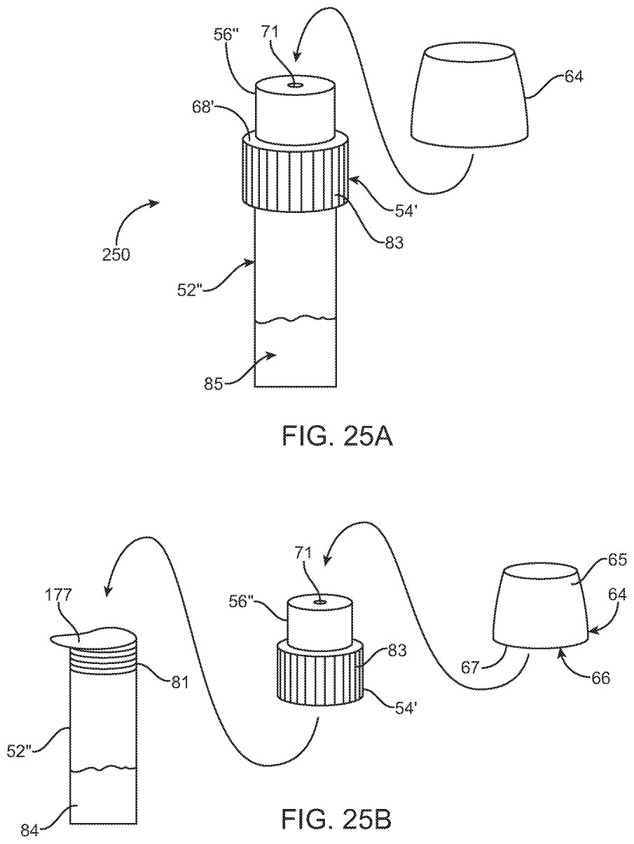

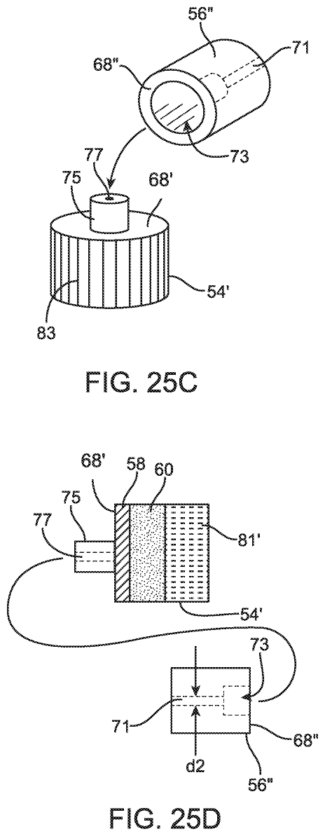

Referring now to attached FIGS. 25a-25d, an extraction vial 250 includes a tubular body 52″, applicator cap 54′, filters 58,60, seal 177 covering an opening of tubular body 52″, liquid buffer 84, porous applicator 56″, and sealing cap 64 as disclosed for extraction vials 50/50′. Extraction vial 250 is configured for preparing a liquid mixture 85 composed of a liquid buffer and a biological specimen to facilitate the determination of the presence and/or amount of one or more targets present in the biological specimen as disclosed above for extraction vials 50, 50′, 150. Extraction vial 250 may be used in a workflow for determining the presence and/or amount of one or more target(s) present in a biological specimen as disclosed for such extraction vials, e.g., with respect to extraction vial 50.

Sealing cap 64 includes a generally tubular body 65 with a cap opening 66. Sealing cap 64 is configured to accommodate porous applicator 56″ therein such that a lip 67 of cap opening 66 engages a distal shoulder 68′ of applicator cap 54′ via a snap-fit or other securing mechanism, e.g., threads, to prevent liquid mixture from exiting extraction vial 250. Applicator cap 54′ engages tubular body 52″ via threads 81,81′ (see for example, FIGS. 25b and 25d) or other securing mechanism (e.g., a snap-fit).

Extraction vial 250 is configured to facilitate equalization of the gas pressure within tubular body 52″ with the gas pressure of the ambient atmosphere following the expulsion of sample liquid 85 from tubular body 52″ into porous applicator 56″. Porous applicator 56″ includes a distal socket 73, a passage 71 having an internal diameter d2, and a shoulder 68″. The shoulder 68″ may be configured to contact shoulder 68′. Internal diameter d2 of passage 71 is typically small enough that capillary forces therein prevent liquid 85 from running out of passage 71 and to instead wick into applicator 56″ for application to a strip as illustrated in attached FIG. 24g. For example, internal diameter d2 may be about 2 mm or less, about 1.5 mm or less, about 1 mm or less, or about 75 microns or less.

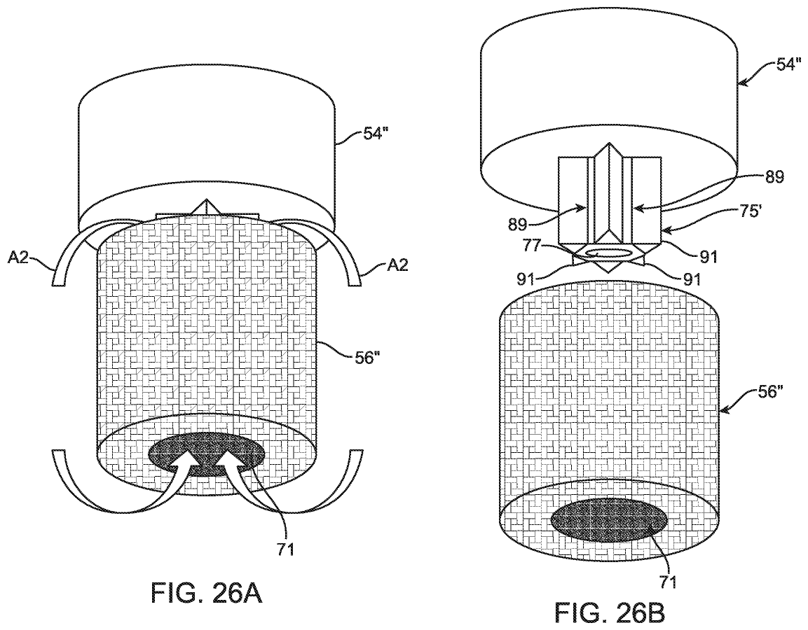

Applicator cap 54′ includes a proximal cap projection 75 sized and configured to be accommodated securely within distal socket 73 of porous applicator 56″. Applicator cap 54′ further includes a cap passage 77 extending through cap projection 75. Distal socket 73 and proximal cap projection 75 are illustrated as cylindrical but could have other cross sections, e.g., polygonal (such as square or triangular), textured (such as a cap projection with knurled surface), splined (such as star-shaped), or combination thereof. With reference to FIGS. 26a-26b, an applicator cap 54″ includes a splined proximal cap projection 75′, which contacts an internal surface of applicator 56″ only at discrete contact points created by surfaces 91 thereby leaving gaps 89 between cap projection 75′ and an internal surface of distal socket 73 of applicator 56″. Gaps 89 facilitate the re-entry of gas (e.g., air) into tubular body 52″ following the expulsion of liquid as discussed below.

Extraction vial 250 can be operated as follows in a workflow for determining the presence and/or amount of one or more target(s) present in a biological specimen as disclosed for extraction vial 50. Once a biological specimen has been obtained, a user positions extraction vial 250 in a non-application orientation in which the liquid buffer 84 gathers adjacent a base of tubular body 52″ and opposite the opening covered by seal 177. The user removes seal 177 to expose the body opening of tubular body 52″ and liquid buffer 84 therein. The user then forms a liquid mixture as disclosed for extraction vial 50.

The user then secures applicator cap 54′ over the body opening of tubular body 52″ so that liquid mixture 85 must pass through filters 58,60 when exiting tubular body 52″ via passage 77 of cap projection 75. When the user is ready to perform a determination for a target(s) present in liquid mixture 85, the user prepares a microfluidic device for operation by a diagnostic reader, e.g., as disclosed extraction vial 50. The user positions extraction vial 250 in the application orientation such that liquid mixture 85 contacts the exposed surface of filter 60 and then begins to pass into and through filters 58,60. Liquid (filtrate) of liquid mixture 85 may pass through the filters and applicator via capillary action. Alternatively, or in combination, a user may increase gas pressure within tubular body 52″ by squeezing external surfaces thereof as disclosed for extraction vial 50 to force liquid through the filters. Liquid mixture 85 passes through filters 58,60, into passage 77 of cap projection 75, exits cap projection 75 and enters passage 71 of porous applicator 56″. Capillary forces within passage 71 prevent liquid mixture 85 from merely flowing out of porous applicator 56″. Instead, liquid mixture 85 wicks into porous applicator 56″, which may have a liquid sufficiency indicator as described for porous applicator 56 of extraction vial 50.

Once sufficient liquid has entered porous applicator 56″, the user may decrease gas pressure within tubular body 52″ e.g., by reducing pressure on external surfaces thereof. Upon reducing the pressure applied to the external surfaces of tubular body 52″, the walls thereof tend to return to an uncompressed state increasing the internal volume of tubular body 52″ and reducing the gas pressure therein. The reduced internal gas pressure causes gas, e.g., air from the ambient atmosphere surrounding extraction vial 250, to be forced into tubular body 52″ via passage 71 of porous applicator 56″ and passage 77 of cap projection 75. The presence of passage 71 of porous applicator 56″ reduces the resistance that would otherwise impede gas reentering tubular body 52″ upon the reduction of gas pressure therein. Although passage 71 is illustrated as an open passage extending through porous applicator 56″, other embodiments are possible. For example, the passage may not extend entirely through the porous applicator but may be closed, e.g., at a distal or proximal end of the porous applicator. In such embodiments, the pathlength for gas to return to the tubular body 52″ (and therefore the resistance to gas return flow) is still less than it would be in the absence of any passage through the porous applicator. As another example, the passage may not be fully open as illustrated for passage 71 but may be a region of reduced density and/or increased porosity of the porous applicator that provides the benefit of reduced resistance for gas return. In some cases, the gas return allows for the gas to enter into the tubular body, and thereby help prevent the liquid sample from being drawn back into the tubular body due to the lower pressure therein.

With sufficient liquid mixture 85 present in porous applicator 56″, the user may then apply liquid sample to the microfluidic strip as described for extraction vials 50,50′,150. Once sufficient liquid (filtrate) of liquid mixture 85 has been applied to the sample application zone, the user repositions extraction vial 250 in the non-application orientation and encloses applicator 56″ with sealing cap 64. The diagnostic reader then operates the microfluidic device to determine one or more targets in the applied liquid, e.g., as disclosed herein for the strip of FIGS. 1, 17, 18, or for any of the microfluidic devices of the '325 Application.

### EXAMPLES

### Example 1: End Fill Application Via Liquid Sample Held by Collection Swab

SARS-CoV 2 virus detection was compared using a top fill microfluidic device having a sample application zone with a port disposed on an upper surface of the device and an end fill microfluidic device having a sample application zone with a port disposed at a periphery of the device (see Embedded FIG. 7, below). The top fill microfluidic device was a microfluidic strip configured and operated for detection of SARS-COV2 virus antigen as disclosed in the '325 Application. Each strip included a first reagent with a detectable fluorescent label and a second reagent with a magnetic particle as disclosed in the '325 Application. The end fill device was prepared from such a top fill device by cutting off the proximal end of the device along a line intersecting the common supply channel (as such term is used in the '325 application) of the microfluidic device such that the common supply channel formed an opening in the proximal periphery of the microfluidic device, the opening forming a sample application port of the microfluidic device.

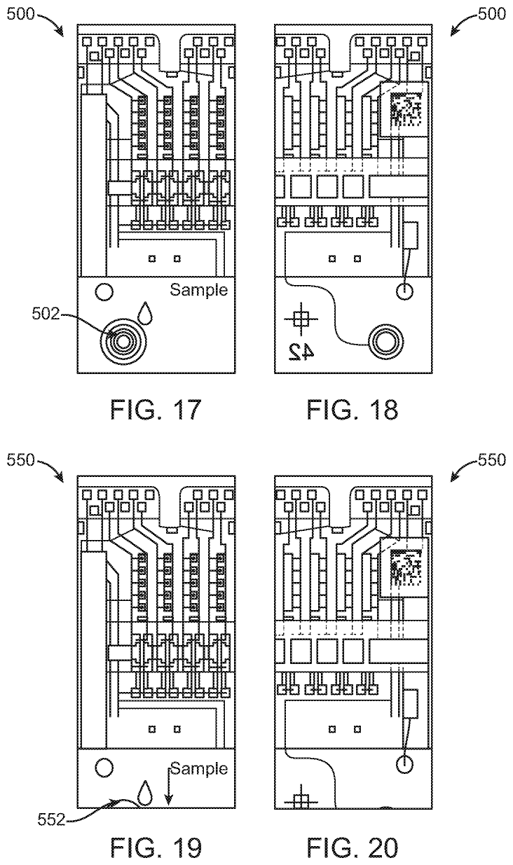

FIGS. 17 and 18 depict a top and bottom view, respectively, of an exemplary top fill microfluidic strip 500, wherein sample is provided via a port located at the top of the device 500.

FIGS. 19 and 20 depict a top and bottom view, respectively, of an exemplary end fill microfluidic strip 550, wherein sample is provided via a port located at a proximal periphery of the end fill strip 550.

A serial dilution of heat inactivated SARS CoV 2 virus was made in a buffer solution based on an original stock concentration Median Tissue Culture Infectious Dose (TCID 50) of 1.51×10⁶. The buffer solution in which dilutions were performed was 400 mM tris, 1 mg/ml BSA. This buffer solution did not cause the virus to lyse. An aliquot of 50 μl of each virus dilution was applied to a swab (swab from MWE, cat no. MW 112) and left for 10 seconds before the swab tip with dilution aliquot was introduced to a tube of extraction buffer for an incubation of 1 min, with the swab agitated during this incubation to combine the dilution buffer and extraction buffer. The volume of extraction buffer in each respective tube was 700 μl (for use with the top fill strip) and 150 μl (for use with the end fill strip). The extraction buffer was configured to lyse virus present in the mixture of the extraction and dilution buffers.

Performing determinations using the top fill strips: For each respective dilution, the swab tip was removed from the tube with 700 μl of extraction buffer, while squeezing the swab tip to elute as much dilution buffer from the swab as possible. After removing the swab tip from the buffer, the cap was applied to the tube. The liquid sample in each tube was applied dropwise to a respective top fill strip through a tube cap with filter as disclosed in U.S. Patent Application No. 63/082,246, titled “Extraction Container”, and filed Sep. 23, 2020, which application is incorporated herein by reference in its entirety. The strip was operated using an instrument as disclosed in the '325 Application except that the determination was performed using only one of the four available channels of the strip. The instrument detected the fluorescence intensity emitted from the detection zone of each strip.

Performing determinations using the end fill strips: For each respective dilution, the swab was removed from the tube with 150 μl of extraction buffer, without squeezing the swab tip to so that the swab tip held liquid sample. The swab tip holding the liquid sample was then applied to the sample application port of a respective end fill strip. Liquid sample from the swab tip flowed into the microfluidic network of the end fill strip via capillary action. Each strip was operated using the same instrument as for the top fill strips, with the determination performed in only one of the available four channels.

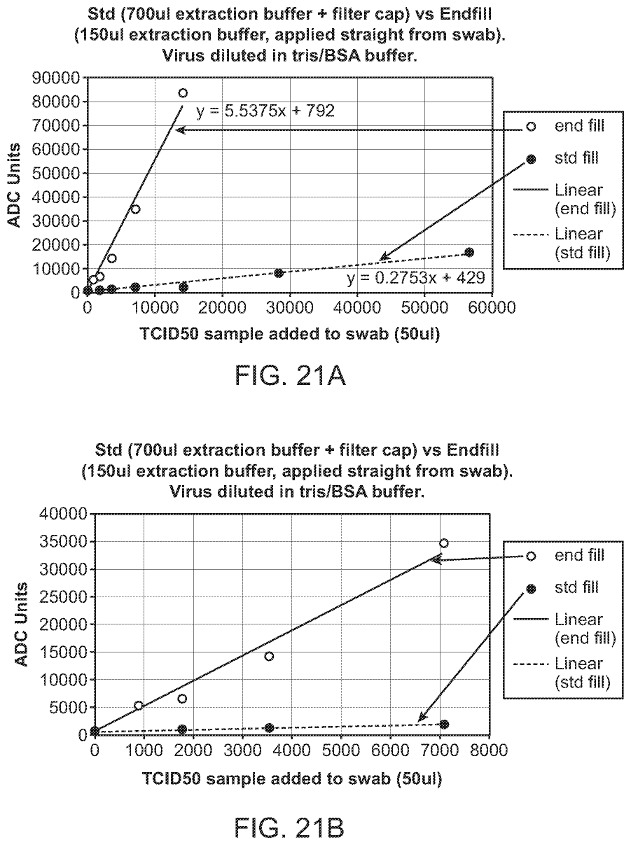

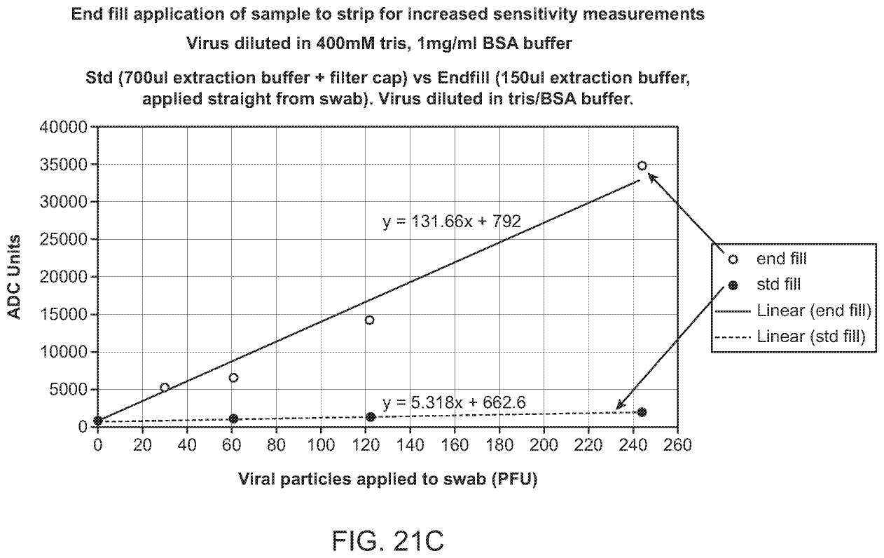

FIGS. 21A-B illustrate data points corresponding to the fluorescence intensity for each respective dilution using the top fill strip (identified as “Std” in the plots) and end fill strip. The data plot in FIG. 21A illustrates fluorescence intensities for the higher concentrations of the viral dilution series and the data plot in FIG. 21B illustrates fluorescence intensities for the lower concentrations of the viral dilution series. In each plot, the data points and fitted line for the end fill strips have a higher slope than the corresponding data points and fitted line for the top fill (std fill) strips.

FIG. 21C illustrates the fluorescence intensities for the lower concentrations of the viral dilution series plotted against the number of virus particles applied to the strip (PFU) on the x axis, rather than the TCID50, with the PFU calculated by converting each TCID50 to PFU by multiplying by 0.69 from Poisson distribution.

At each end of the dilution series, the end fill strip (with 150 μl extraction buffer) exhibits greater sensitivity than the top fill (std fill) strip (700 μl extraction buffer). Applying the virus sample to the swab unlysed and allowing the virus to be lysed by a 1 min incubation with 150 ul extraction buffer does not impede the performance of the assay. In fact, the assay performs better with this method, possibly due to the addition of BSA in the dilution buffer potentially preventing/reducing the loss of virus to the tube surfaces during the serial dilution. In another experiment (not shown) the virus was diluted in PBS alone and the signals of both the end fill and standard application dropped, especially at the low viral concentrations, reinforcing the idea of possible viral loss to the tubes in the absence of a blocking agent (such as BSA).

The difference in dilution volume alone (150 μl vs 700 μl) is not sufficient to explain the increased performance of the end fill methodology. Perhaps the filter used in the cap by the standard application method is also binding so some analyte to reduce the concentration applied to the strip further It is thought that although a direct end fill of a strip from a swab does not use a filter, this will not affect the performance of the assay negatively as only solubilized material may be drawn into the strip by capillary action with the swab material itself potentially acting as a filter.

### Additional Embodiments

Disclosed herein, in some aspects, is a method, comprising: applying a liquid sample held by a tip of a collection swab to a sample introduction port of a microfluidic device.

In some embodiments, the microfluidic device comprises a microfluidic network in fluidic communication with the sample introduction port and the applying the liquid sample held by the tip of the collection swab to the sample introduction port comprises drawing at least some of the liquid sample held by the tip of the collection swab from the tip through the sample introduction port and into at least a portion of the microfluidic network. In some embodiments, the method comprises performing the drawing the at least some liquid sample by capillary action within the sample introduction port and/or the at least a portion of the microfluidic network. In some embodiments, the drawing the sample through the sample introduction port and into at least a portion of the microfluidic network comprises flowing at least some of the liquid sample drawn from the tip of the collection swab along at least a portion of the microfluidic network. In some embodiments, the flowing the at least some of the liquid sample drawn from the tip of the collection swab along at least a portion of the microfluidic network is performed by capillary action. In some embodiments, the microfluidic network includes a capillary stop and the flowing the at least some of the liquid sample drawn from the tip of the collection swab along at least a portion of the microfluidic network comprises stopping the flowing when a distal liquid-gas interface of the liquid sample reaches the capillary stop. In some embodiments, the capillary stop comprises a vent in gaseous communication with an ambient atmosphere surrounding the microfluidic device.

In some embodiments, the method comprises solubilizing, with the liquid sample drawn from the tip of the collection swab through the sample introduction port and into at least a portion of the microfluidic network, at least one reagent disposed within the at least a portion of the microfluidic network. In some embodiments, the at least one reagent comprises a first reagent, the first reagent comprising a first portion configured to bind a target indicative of a pathogen and a second portion comprising an optical label. In some embodiments, the at least one reagent comprises a second reagent, the second reagent comprising a first portion configured to bind the target, e.g., in a sandwich relationship with the target and the first reagent, and a second portion comprising a magnetic particle.

In some embodiments, the method further comprises flowing the at least some liquid including the solubilized at least one reagent along the microfluidic network beyond the capillary stop. In some embodiments, the flowing the at least some liquid including the solubilized at least one reagent along the microfluidic network beyond the capillary stop comprises decreasing a pressure of the gas of the distal liquid-gas interface as compared to a pressure of the ambient atmosphere. In some embodiments, the decreasing the pressure of the gas comprises increasing a volume occupied by the gas of the distal liquid-gas interface within the microfluidic network. In some embodiments, the increasing the volume occupied by the gas comprises increasing a height of the microfluidic channel network at a location occupied by the gas of the distal liquid-gas interface. In some embodiments, the increasing the height comprises rotating an eccentric member, e.g., a wheel, having a periphery disposed in contact with an exterior of the microfluidic device overlying the location occupied by the gas of the distal liquid-gas interface.

In some embodiments, the flowing the at least some applied liquid including the solubilized at least one reagent along the microfluidic network beyond the capillary stop comprises flowing the at least some applied liquid including the solubilized reagent through a localized magnetic field. In some embodiments, the flowing the at least some liquid including the solubilized reagent through a localized magnetic field within the microfluidic network comprises retaining at least the second reagent, including second reagent bound to the target, within a detection zone defined by the localized magnetic field.

In some embodiments, the method comprises generating the localized magnetic field from a permanent magnet disposed adjacent the microfluidic device. In some embodiments, the microfluidic device is disposed in an operatively secure state within an instrument comprising the permanent magnet and the permanent magnet is disposed in a fixed, e.g., operatively immovable, position with respect to the microfluidic device when in the operatively secure state. In some embodiments, the instrument comprises a light source and the method further comprises illuminating the detection zone with light from the light source and detecting light emitted by the detectable label of the first reagent bound to the target and present in the detection zone. In some embodiments, the method further comprises detecting the presence or amount of the detectable label present in the detection zone and determining the presence or amount of the target present in the liquid sample based on the detected detectable label.

In some embodiments, the liquid sample comprises a mixture of a liquid buffer and a biological specimen, e.g., a nasal, salivary, throat, nasopharyngeal, mid turbinate, urine, or vaginal specimen, collected from a mammal, e.g., a human being. In some embodiments, the method comprises, prior to the step of applying the liquid sample, forming the liquid sample by one or more steps including receiving the collection swab, the tip of the collection swab having been used to collect the biological specimen, and introducing the tip of the collection swab into the liquid buffer. In some embodiments, the method comprises, after the step of introducing the tip of the collection swab into the buffer and prior to the step of applying the liquid sample, removing the swab tip from the liquid buffer, the removed swab tip holding the liquid sample. In some embodiments, the total volume of liquid buffer into which the collection swab is introduced is about 225 microliters or less, 200 microliters or less, about 175 microliters or less, about 150 microliters or less, or about 125 microliters or less. In some embodiments, the liquid buffer comprises a blocking agent, e.g., a protein-based blocking agent, e.g., a protein-based blocking agent such as bovine serum albumin.

In some embodiments, the tip of the collection swab (i) comprises a plurality of fibers, e.g., as a flocked swab tip or a spun fiber swab tip, (ii) comprises a sponge or foam, (iii) is a sintered swab tip, (iv) is a three-dimensional printed swab tip, or (v) includes a combination of two or more swab tips of clauses (i) (iv).

In some embodiments, the introduction port is disposed at or adjacent a periphery of the microfluidic device. In some embodiments, the introduction port is disposed at a periphery of the microfluidic device. In some embodiments, the periphery of the microfluidic device defines a peripheral face and the introduction port comprises an opening disposed in the peripheral face. In some embodiments, the introduction port is arranged as an end fill introduction port.

In some embodiments, the method further comprises determining the presence and/or amount of the target present in the liquid sample that has flowed along the at least a portion of the microfluidic network.

In some embodiments, the microfluidic network consists essentially of a single microchannel.

In some embodiments, the step of applying comprises moving the tip of the collection swab holding the liquid sample from a non-application state to an application state, wherein in the non-application state the sample liquid held by the tip of the swab is not in fluidic communication with the sample introduction port of the microfluidic device and in the application state the sample liquid held by the tip of the swab is in fluidic communication with the sample introduction port.

In some embodiments, the method further comprises, prior to the step of applying, using the tip of the collection swab to collect the biological specimen from the mammal. In some embodiments, the collection swab including the tip thereof is mechanically and fluidically separated from the microfluidic device during the step of using the tip of the collection swab to collect the biological specimen.

In some embodiments, the method is performed without combining, either concurrently with or subsequently to the step of applying, the liquid sample held by the tip of the collection swab and applied to the sample introduction port of the microfluidic device with another liquid.

In some embodiments, during the method, the microfluidic device is at least essentially free, e.g., is free, of any liquid other than the liquid sample drawn from the tip of the collection swab through the sample introduction port and into at least a portion of the microfluidic network.

In some embodiments, the method is performed without applying a liquid other than the liquid sample held by the tip of the collection swab to the sample introduction port of the microfluidic device.

In some embodiments, the method is performed without introducing into the microfluidic device a liquid other than the liquid sample held by the tip of the collection swab and drawn through the sample introduction port and into at least a portion of the microfluidic network.

In some embodiments, the microfluidic device lacks any reservoir for a liquid and/or lacks any port for introducing a liquid to the microfluidic device other than the sample introduction port.

In some embodiments, the step of applying is performed after the microfluidic device has been inserted into a microfluidic device introduction port of an instrument configured to operate the microfluidic device to determine the presence and/or amount of a target, e.g., a target indicative of the presence of a pathogen, present in the liquid sample applied to the sample introduction port.

In some embodiments, the microfluidic network of the microfluidic device is formed of non-porous material.

In some embodiments, the total volume of the liquid sample drawn from the tip of the collection swab through the sample introduction port and into at least a portion of the microfluidic network is about 6 μl or less, about 5 μl or less, about 4 μl or less, about 3.5 μl or less, or about μl or less.

In some embodiments, the total volume of the liquid sample drawn from the tip of the collection swab through the sample introduction port and into at least a portion of the microfluidic network is at least about 1 μl, at least about 2 μl, or at least about 2.5 μl.

For any method disclosed herein, in some embodiments, the method further comprises, after the step of applying, separating the tip of the collection swab mechanically and/or fluidically from the sample introduction port of the microfluidic device. For example, the separating may be performed after applying the sample but prior to one or more of the sample having substantially combined with reagents within the microfluidic device, the sample having entered a detection zone of the microfluidic device, and/or prior to the detection of signals from the detection zone, e.g., optical or electrochemical signals, indicative of the presence and/or amount of target present in the sample. In some embodiments, the tip of the collection swab or other applicator remains mechanically free of the microfluidic device such that the collection swab or other applicator is not integrated with, restrained with respect to, or secured with respect to the microfluidic device. Accordingly, a user may manually position the tip of the collection swab or other applicator in fluidic contact with the application port of the microfluidic device and then remove the collection swab or other applicator from the fluidic contact without having to release a securing mechanism or other mechanical means restraining movement of the swab/applicator with respect to the microfluidic device.

In some embodiments, the applying is performed without substantially compressing the tip of the collection swab. In some embodiments, the tip of the collection swab has a total volume, including the liquid sample, of V immediately prior to the applying the liquid sample to the sample application port and, during the applying, the total volume of the tip remains at least about 0.7×V, at least about 0.8×V, at least about 0.9×V, at least about 0.95×V, or at least about 0.975×V.

In some embodiments, substantially all of the liquid sample that enters the microfluidic network from the collection swab tip during the applying is drawn therein by capillary action within the sample introduction port and/or the at least a portion of the microfluidic network. In some embodiments, at least about 75%, at least about 85%, at least about 90%, at least about 95%, at least about 97.5% or essentially all of the liquid sample that enters the microfluidic network from the collection swab tip during the applying is drawn therein by capillary action within the sample introduction port and/or the at least a portion of the microfluidic network.

In some embodiments, the applying is performed without substantially increasing a pressure experienced by the liquid sample held by the tip of the collection swab as compared to a pressure experienced prior to the applying. In some embodiments, the pressure experienced by the liquid sample held by the tip of the collection swab during the applying is about 1.2×P or less, about 1.15×P or less, about 1.1×P or less, about 1.05×P or less, or essentially the same as P, wherein P is the gas pressure of the ambient atmosphere surrounding the tip of the collection swab.

In some embodiments, the collection swab is a nasopharyngeal collection swab, oral swab, oropharyngeal collection swab, or vaginal collection swab.

Disclosed herein, in some aspects, is a microfluidic device, comprising: a microfluidic channel network comprising a sample application port, first time gate channel, a reagent zone (e.g., a reagent chamber), a measurement zone (e.g., a measurement chamber or a detection zone as described herein), and a second time gate channel, wherein the first and second time gate channels are spaced apart by the reagent zone and measurement zone.

Disclosed herein, in some aspects, is a method, comprising: a) providing a microfluidic device comprising a microfluidic channel network comprising a sample application port, a first time gate channel, a reagent zone, a measurement zone, and a second time gate channel; b) introducing a sample liquid to the sample application port of the microfluidic channel network; c) flowing the sample liquid by capillary action along the first time gate channel; d) flowing the sample through the reagent zone and measurement zone and capturing one or more targets present in the sample liquid with one or more reagents present in the reagent zone; and e) flowing the sample from the reagent zone and measurement zone through the second time gate channel.

Disclosed herein, in some aspects, is a method comprising: a) introducing a collection swab tip into a liquid buffer, which liquid buffer optionally includes a blocking agent such as a protein-based blocking agent such as bovine serum albumin, the collection swab tip comprising a liquid sample, e.g., a nasal, salivary, throat, mid turbinate, nasopharyngeal, or vaginal sample, collected from a mammal, e.g., a human being; b) removing the collection swab tip from the buffer, the removed collection swab tip holding a mixture of the liquid sample and buffer; c) contacting a sample application zone of a microfluidic device, which sample application zone is optionally disposed at a periphery of the microfluidic device, with the removed swab tip and permitting an amount of the mixture to flow by capillary action from the swab tip through the sample application zone and into a microfluidic network of the microfluidic device; d) combining at least some of the mixture that has flowed into the microfluidic network with one or more mobilizable reagents disposed within the microfluidic network; and e) determining the presence or amount of one or more targets in the mixture based upon an interaction within the microfluidic network of each of the one or more targets and at least one of the reagents, wherein each of the targets is indicative of the presence of a respective pathogen present in the liquid sample.

Disclosed herein, in some aspects, is a method comprising: a) introducing a collection swab tip into a mixture comprising a liquid buffer, which liquid buffer optionally includes a blocking agent such as a protein-based blocking agent such as bovine serum albumin, and a liquid sample, e.g., a nasal, salivary, throat, mid turbinate, nasopharyngeal, or vaginal sample, collected from a mammal, e.g., a human being; b) removing the collection swab tip from the buffer, the removed collection swab tip holding a mixture of the liquid sample and buffer; c) contacting a sample application zone of a microfluidic device, which sample application zone is optionally disposed at a periphery of the microfluidic device, with the removed swab tip and permitting an amount of the mixture to flow by capillary action from the swab tip through the sample application zone and into a microfluidic network of the microfluidic device; d) combining at least some of the mixture that has flowed into the microfluidic network with one or more mobilizable reagents disposed within the microfluidic network; and e) determining the presence or amount of one or more targets in the mixture based upon an interaction within the microfluidic network of each of the one or more targets and at least one of the reagents, wherein each of the targets is indicative of the presence of a respective pathogen present in the liquid sample.

Disclosed herein, in some aspects, is a method comprising: a) introducing a collection swab tip into a liquid buffer, which liquid buffer optionally comprises a blocking agent such as a protein-based blocking agent such as bovine serum albumin, the collection swab tip comprising a liquid sample, e.g., a nasal, salivary, throat, mid turbinate, nasopharyngeal, or vaginal sample, collected from a mammal, e.g., a human being, the buffer having a total volume of about 225 microliters or less, 200 microliters or less, about 175 microliters or less, about 150 microliters or less, or about 125 microliters or less; b) removing the collection swab tip from the buffer, the removed collection swab tip holding a mixture of the liquid sample and buffer; c) contacting a sample application zone of a microfluidic device, which sample application zone is optionally disposed at a periphery of the microfluidic device, with the removed swab tip and permitting an amount of the mixture to flow by capillary action from the swab tip through the sample application zone and into a microfluidic network of the microfluidic device; and d) determining the presence or amount of one or more targets in the mixture based upon an interaction within the microfluidic network of each of the one or more targets and at least one of the reagents, wherein each of the targets is indicative of the presence of a respective pathogen present in the liquid sample.

Disclosed herein, in some aspects, is a method comprising: a) introducing a collection swab tip into mixture comprising a liquid buffer, which liquid buffer optionally comprises a blocking agent such as a protein-based blocking agent such as bovine serum albumin and a liquid sample, e.g., a nasal, salivary, throat, mid turbinate, nasopharyngeal, or vaginal sample, collected from a mammal, e.g., a human being, the mixture having a total volume of about 225 microliters or less, 200 microliters or less, about 175 microliters or less, about 150 microliters or less, or about 125 microliters or less; b) removing the collection swab tip from the buffer, the removed collection swab tip holding an amount of the mixture; c) contacting a sample application zone of a microfluidic device, which sample application zone is optionally disposed at a periphery of the microfluidic device, with the removed swab tip and permitting an amount of the mixture to flow by capillary action from the swab tip through the sample application zone and into a microfluidic network of the microfluidic device; and d) determining the presence or amount of one or more targets in the mixture based upon an interaction within the microfluidic network of each of the one or more targets and at least one of the reagents, wherein each of the targets is indicative of the presence of a respective pathogen present in the liquid sample.

Disclosed herein, in some aspects, is a method, comprising: a) providing a collection swab having a collection swab tip, the collection swab tip having been used to collect a biological specimen from a mammal, e.g., a human being, the biological specimen comprising at least one of a nasal, salivary, throat, nasopharyngeal, mid turbinate, urine, or vaginal specimen; b) introducing the collection swab tip with biological specimen into a liquid buffer; c) removing the collection swab tip from the liquid buffer, the collection swab tip holding a liquid mixture of the biological specimen and the liquid buffer; d) applying the collection swab tip removed from the liquid buffer to a sample application port of a microfluidic device whereupon liquid mixture from the collection swab tip flows by capillary action through the sample application port and into a microfluidic network of the microfluidic device; e) combining the liquid mixture with at least one reagent within the microfluidic network, the at least one reagent configured to interact with, e.g., bind to, a target present in the liquid mixture, the target indicative of the presence of a pathogen in the biological specimen; and f) determining the presence and/or amount of the pathogen in the liquid mixture based upon the interaction between the at least one reagent and target.

Disclosed herein, in some aspects, is a method, comprising: a) introducing a collection swab tip of a collection swab into liquid mixture comprising a liquid buffer and a biological specimen from a mammal, e.g., a human being, the biological specimen comprising at least one of a nasal, salivary, throat, nasopharyngeal, mid turbinate, urine, or vaginal specimen; b) removing the collection swab tip from the liquid mixture, the collection swab tip holding an amount of the liquid mixture; c) applying the collection swab tip removed from the liquid mixture to a sample application port of a microfluidic device whereupon liquid mixture from the collection swab tip flows by capillary action through the sample application port and into a microfluidic network of the microfluidic device; d) combining the liquid mixture with at least one reagent within the microfluidic network, the at least one reagent configured to interact with, e.g., bind to, a target present in the liquid mixture, the target indicative of the presence of a pathogen in the biological specimen; and e) determining the presence and/or amount of the pathogen in the liquid mixture based upon the interaction between the at least one reagent and target.

Disclosed herein, in some aspects, is a method comprising: a) introducing a collection swab tip into a liquid buffer, which liquid buffer optionally includes a blocking agent such as a protein-based blocking agent such as bovine serum albumin, the collection swab tip comprising a liquid sample, e.g., a nasal, salivary, throat, mid turbinate, nasopharyngeal, or vaginal sample, collected from a mammal, e.g., a human being; b) removing the collection swab tip from the buffer; c) after introducing the collection swab tip into the liquid buffer, introducing a second swab tip into the liquid buffer; d) removing the second swab tip from the liquid buffer, the second swab tip holding a liquid mixture of the biological specimen and the liquid buffer; e) contacting a sample application zone of a microfluidic device with the removed second swab tip and permitting an amount of the liquid mixture to flow by capillary action from the second swab tip through the sample application zone and into a microfluidic network of the microfluidic device, wherein the sample application zone is optionally disposed at a periphery of the microfluidic device; f) combining at least some of the liquid mixture that has flowed into the microfluidic network with one or more mobilizable reagents disposed within the microfluidic network; and g) determining the presence or amount of one or more targets in the liquid mixture based upon an interaction within the microfluidic network of each of the one or more targets and at least one of the reagents, wherein each of the targets is indicative of the presence of a respective pathogen present in the liquid sample.

Disclosed herein, in some aspects, is a method comprising: a) introducing a collection swab tip into a liquid buffer, which liquid buffer optionally comprises a blocking agent such as a protein-based blocking agent such as bovine serum albumin, the collection swab tip comprising a liquid sample, e.g., a nasal, salivary, throat, mid turbinate, nasopharyngeal, or vaginal sample, collected from a mammal, e.g., a human being, the buffer having a total volume of about 225 microliters or less, 200 microliters or less, about 175 microliters or less, about 150 microliters or less, or about 125 microliters or less; b) removing the collection swab tip from the liquid buffer; c) after introducing the collection swab tip into the liquid buffer, introducing a second swab tip into the liquid buffer; d) removing the second swab tip from the liquid buffer, the second swab tip holding a liquid mixture of the biological specimen and the liquid buffer; e) contacting a sample application zone of a microfluidic device with the removed second swab tip and permitting an amount of the liquid mixture to flow by capillary action from the second swab tip through the sample application zone and into a microfluidic network of the microfluidic device, wherein the sample application zone is optionally disposed at a periphery of the microfluidic device; and f) determining the presence or amount of one or more targets in the mixture based upon an interaction within the microfluidic network of each of the one or more targets and at least one of the reagents, wherein each of the targets is indicative of the presence of a respective pathogen present in the liquid sample.

Disclosed herein, in some aspects, is a method, comprising: a) providing a collection swab having a collection swab tip, the collection swab tip having been used to collect a biological specimen from a mammal, e.g., a human being, the biological specimen comprising at least one of a nasal, salivary, throat, nasopharyngeal, mid turbinate, urine, or vaginal specimen; b) introducing the collection swab tip with biological specimen into a liquid buffer; c) removing the collection swab tip from the liquid buffer, the collection swab tip holding a liquid mixture of the biological specimen and the liquid buffer; d) applying the collection swab tip removed from the liquid buffer to a sample application port of a microfluidic device whereupon liquid mixture from the collection swab tip flows by capillary action through the sample application port and into a microfluidic network of the microfluidic device; e) combining the liquid mixture with at least one reagent within the microfluidic network, the at least one reagent configured to interact with, e.g., bind to, a target present in the liquid mixture, the target indicative of the presence of a pathogen in the biological specimen; and f) determining the presence and/or amount of the pathogen in the liquid mixture based upon the interaction between the at least one reagent and target.

Disclosed herein, in some aspects, is a method, comprising: a) forming a liquid mixture comprising a biological specimen from a mammal, e.g., a human being, the biological specimen comprising at least one of a nasal, salivary, throat, nasopharyngeal, mid turbinate, urine, or vaginal specimen, and a liquid buffer; b) introducing a swab tip of a collection swab into the liquid mixture; c) removing the swab tip of the collection swab from the liquid buffer, the swab tip holding an amount of the liquid mixture; d) applying the swab tip removed from the liquid mixture to a sample application port of a microfluidic device whereupon liquid mixture from the collection swab tip flows by capillary action through the sample application port and into a microfluidic network of the microfluidic device; e) combining the liquid mixture with at least one reagent within the microfluidic network, the at least one reagent configured to interact with, e.g., bind to, a target present in the liquid mixture, the target indicative of the presence of a pathogen in the biological specimen; and f) determining the presence and/or amount of the pathogen in the liquid mixture based upon the interaction between the at least one reagent and target.

Described herein, in some aspects, is a method includes applying a liquid sample held by a porous member to a sample introduction port of a microfluidic device. The microfluidic device may include a microfluidic network in fluidic communication with the sample introduction port and the method may further include flowing at least some of the liquid sample applied to the sample introduction port along at least a portion of the microfluidic network. The step of flowing the at least some of the applied liquid sample along the at least a portion of the microfluidic network may be performed by capillary action. For example, upon applying the liquid sample to the sample introduction port, capillary action may draw the at least some of the applied liquid sample from the liquid sample held by the porous member into the sample introduction port and along the at least a portion of the microfluidic network. The total volume of the liquid sample drawn from the porous member through the sample introduction port and into at least a portion of the microfluidic network may be, e.g., about 10 μl or less, about 6 μl or less, about 5 μl or less, about 4 μl or less, about 3.5 μl or less, or about 3.0 μl or less. The total volume of the liquid sample drawn from the porous member through the sample introduction port and into at least a portion of the microfluidic network may be, e.g., at least about 1 μl, at least about 2 μl, or at least about 2.5 μl.

In embodiments of any of the foregoing methods, the microfluidic network includes a capillary stop and the step of flowing the at least some of the applied liquid sample along the at least a portion of the microfluidic network includes stopping the flowing when a distal liquid-gas interface of the liquid sample reaches the capillary stop. Prior to the stopping the flowing, the flowing of the liquid sample along the at least a portion of the microfluidic network may be performed substantially, e.g., essentially entirely, by capillary action. For example, prior to the stopping the flowing, the flowing may be performed without applying a motive force to the liquid sample other than the motive force created by capillary action within the microfluidic network. The capillary stop may include a vent in gaseous communication with an ambient atmosphere surrounding the microfluidic device.

In embodiments of any of the foregoing methods, the step of flowing the at least some of the applied liquid sample along the at least a portion of the microfluidic network includes solubilizing with the at least some of the applied liquid sample, at least one reagent disposed within the at least a portion of the microfluidic network. The solubilizing wets and/or mobilizes the reagents from a dry state permitting the reagents to interact with the liquid sample. The at least one reagent may include a first reagent, the first reagent including a first portion configured to bind a target indicative of a pathogen and a second portion comprising an optical label. The at least one reagent may include a second reagent, the second reagent including a first portion configured to bind the target, e.g., in a sandwich relationship with the target and the first reagent, and a second portion comprising a magnetic particle. When solubilized, such reagents are typically mobilized by the liquid sample such that the reagents may be moved by the liquid sample along the microfluidic network. The step of solubilizing may be initiated prior to or concurrently with a step of stopping the flowing the at least some applied liquid sample when the distal liquid-gas interface of the liquid sample reaches a capillary stop. For example, the at least one reagent may be disposed within the microfluidic network in a location that is proximal to (upstream of) a capillary stop such that the least some applied liquid sample contacts and begins to solubilize the at least one reagent prior to the distal liquid-gas interface of the liquid sample reaching the capillary stop. The at least one reagent, e.g., the first and/or second reagent, may be deposited within the microfluidic network, disposed within the microfluidic network, and/or of a composition as disclosed in the '325 Application.

In embodiments of any of the foregoing methods, the method further includes flowing the at least some applied liquid sample including the solubilized at least one reagent along the microfluidic network beyond the capillary stop. The flowing the at least some of the applied liquid sample including the solubilized at least one reagent along the microfluidic network beyond the capillary stop may include subjecting the at least some applied liquid to a motive force other than capillary forces, e.g., by decreasing a pressure of the gas of the distal liquid-gas interface as compared to a pressure of the ambient atmosphere. The decreasing the pressure of the gas may include increasing a volume occupied by the gas of the distal liquid-gas interface within the microfluidic network. The increasing the volume occupied by the gas may include increasing a height of the microfluidic channel network at a location occupied by the gas of the distal liquid-gas interface. The step of decreasing the pressure of the gas may be performed using the system and/or methods disclosed in the '325 Application. Alternatively, the step of increasing the height may include rotating an eccentric wheel having a periphery disposed in direct or indirect (e.g., via a lever arm) contact with an exterior surface of the microfluidic device overlying the location occupied by the gas of the distal liquid-gas interface.

In embodiments of any of the foregoing methods, the flowing the at least some applied liquid including the solubilized at least one reagent along the microfluidic network beyond the capillary stop includes flowing the at least some applied liquid including the solubilized reagent through a localized magnetic field. The flowing the at least some of the applied liquid sample including the solubilized reagent through a localized magnetic field within the microfluidic network may include retaining at least the second reagent, including second reagent bound to the target, within a detection zone defined by the localized magnetic field. For example, the method may include retaining a sandwich complex including the first reagent bound to the target, the target, and the second reagent also bound to the target. The method may include generating the localized magnetic field from a permanent magnet disposed adjacent the microfluidic device. The microfluidic device may be disposed in an operatively secure state within an instrument including the permanent magnet, with the permanent magnet disposed in a fixed, e.g., operatively immovable, position with respect to the microfluidic device when in the operatively secure state. The instrument may include a light source and the method may further include illuminating the detection zone with light from the light source and detecting light emitted by the detectable label of the first reagent bound to the target and present in the detection zone.

In any of the foregoing methods, the liquid sample may include, or consist essentially of, a mixture of a liquid buffer and a biological specimen, e.g., a nasal, salivary, throat, nasopharyngeal, mid turbinate, or vaginal specimen, collected from a mammal, e.g., a human being. In any of the embodiments, the liquid buffer may be an extraction buffer configured to lyse cells present in the biological specimen. Alternatively, the liquid buffer may be configured to preserve cells intact, without lysing or otherwise releasing the contents thereof. The liquid buffer may include a blocking agent, e.g., a protein-based blocking agent, e.g., a protein-based blocking agent such as bovine serum albumin.

In embodiments of any of the foregoing methods, the porous member may be a tip of a collection swab. The method may include, prior to the step of applying the liquid sample, forming the liquid sample by one or more steps including receiving the collection swab, the tip of the collection swab having been used to collect the biological specimen, and introducing the tip of the collection swab into the liquid buffer. The method may include, after the step of introducing the tip of the collection swab into the buffer and prior to the step of applying the liquid sample to the application port of the microfluidic device, removing the swab tip from the liquid buffer, the removed swab tip holding the liquid sample. The total volume of liquid buffer into which the collection swab is introduced may be about 225 microliters or less, 200 microliters or less, about 175 microliters or less, about 150 microliters or less, or about 125 microliters or less.

In any of the foregoing methods, except those embodiments set forth in the immediately preceding paragraph in which the liquid sample includes, or consists essentially of, a mixture of a liquid buffer and a biological specimen, the liquid sample may consist essentially of or consist of a biological specimen, e.g., a nasal, salivary, throat, nasopharyngeal, mid turbinate, or vaginal specimen, collected from a mammal, e.g., a human being. For example, the method may include, prior to the step of applying the liquid sample, contacting the porous member, e.g., a collection swab tip with saliva, e.g., by inserting the porous member, e.g., a collection swab tip into the oral cavity of the mammal. The porous member accumulates oral fluid as a salivary sample which forms the liquid sample that is applied to the sample application zone of the microfluidic device using the porous member.

Any of the foregoing methods may include, prior to the step of applying, collecting the biological specimen from the mammal. During the step of collecting, the porous member may be mechanically and fluidically separated from the microfluidic device including the application port thereof. For example, if the porous member is a tip of a collection swab, the tip of the collection swab may be mechanically and fluidically separated from the microfluidic device including the application port thereof during the collecting.

In any of the foregoing methods, in which the porous member is a tip of a collection swab the tip of the collection swab may (i) include a plurality of fibers, e.g., as a flocked swab tip or a spun fiber swab tip, (ii) include a sponge or foam, (iii) be a sintered swab tip, (iv) be a three-dimensional printed swab tip, or (v) include a combination of two or more swab tips of clauses (i)-(iv).

In any of the foregoing embodiments, the introduction port may be disposed at or adjacent a periphery of the microfluidic device. The periphery of the microfluidic device may define a peripheral face and the introduction port may include an opening disposed in the peripheral face. The introduction port may be arranged as an end fill introduction port.

In embodiments of any of the foregoing embodiments, the method further includes determining the presence and/or amount of the target present in the liquid sample that is held by the porous member applied to the application portion of the microfluidic device, e.g., determining the presence and/or amount of the target present in the liquid sample that flowed along the at least a portion of the microfluidic network.

In any of the foregoing methods, the microfluidic network may consist essentially of a single microchannel. In any of the foregoing methods, the channel of the microfluidic device may be defined by one or more non-porous impermeable substrates, e.g., the microfluidic device may exclude a porous membrane through which sample flows.

In any of the foregoing methods, the step of applying may include moving the tip of the porous member holding the liquid sample from a non-application state to an application state, wherein in the non-application state the sample liquid held by the porous member is not in fluidic communication with the sample introduction port of the microfluidic device and in the application state the sample liquid held by the porous member is in fluidic communication with the sample introduction port. For example, a user may use the porous member, e.g., the tip of a collection swab to collect a biological specimen from a subject, such as a mammal, e.g., a human being. During the process of collecting the biological specimen, e.g., a nasopharyngeal specimen, the porous member, e.g., the collection swab including tip are not in fluidic or mechanical communication with the application port of the microfluidic device. Subsequently, when the porous member is applied to the application port, the porous member, e.g., the tip of a collection swab enters an application state with the porous member being in fluidic communication with the application port. As another example, a user may combine such a biological specimen with a buffer to produce a combination of the biological specimen and buffer. During the process of combining the biological specimen and buffer, a container in which the combination is formed is not in fluidic communication with the application port of the microfluidic device. An amount of the combination of specimen and buffer may be collected using a porous member, e.g., a tip of a collection swab, during which the collection swab and porous member are not in fluidic or mechanical communication with the microfluidic device. Subsequently, to apply the combination of specimen and buffer to the sample application port, the porous member enters an application state with the porous member being in fluidic communication with the sample application port. As an alternative to applying the combination of specimen and buffer via a tip of a collection swab, the applying may be performed using a porous member that is integral with the container in which the combination is formed. For example, the container may be any of the containers disclosed herein in which the cap includes a porous member into which the combination of specimen and buffer may be expressed. The porous member retains the combination to prevent inadvertent loss of liquid (e.g., via dripping) but permits the liquid to be drawn via capillary action from the porous member into the microfluidic device.

In any of the foregoing methods, the method may be performed without combining, either concurrently with or subsequently to the step of applying, the liquid sample or biological specimen held by the porous member and applied to the sample introduction port of the microfluidic device with another liquid.

In any of the foregoing methods, during the performance of the method, the microfluidic device may be at least essentially free, e.g., is free, of any liquid other than the liquid sample drawn from the porous member through the sample introduction port and into at least a portion of the microfluidic network. By essentially free, it is meant that the volume of liquid in the microfluidic device, other than the liquid sample drawn therein from the collection swab tip, is less than about 5% of the volume of the liquid sample drawn therein.

In any of the foregoing methods, the method may be performed without applying a liquid other than the liquid sample or biological specimen held by the porous member to the sample introduction port of the microfluidic device. The sample introduction port may be the only route by which liquids are introduced, e.g., the only route by which liquids can be introduced, to the microfluidic network during operation of the microfluidic device.

In any of the foregoing methods, the method may be performed without introducing into the microfluidic device a liquid other than the liquid sample held by the porous member and drawn through the sample introduction port and into at least a portion of the microfluidic network.

In any of the foregoing methods, the microfluidic device may lack any reservoir for a liquid and/or lacks any port for introducing a liquid to the microfluidic device other than the sample introduction port.

In any of the foregoing methods, the step of applying may be performed after the microfluidic device has been inserted into a microfluidic device introduction port of an instrument configured to operate the microfluidic device to determine the presence and/or amount of a target, e.g., a target indicative of the presence of a pathogen, present in the liquid sample applied to the sample introduction port.

In any of the foregoing methods, the porous member may be mechanically and/or fluidically separated from the sample introduction port of the microfluidic device after applying the liquid sample to the sample introduction port. For example, porous member, e.g., the collection swab with tip may be discarded or at least the tip may be preserved to permit further analysis.

In embodiments, a microfluidic device includes a substrate defining a periphery, a microfluidic network including a sample introduction port and a reagent zone, wherein the sample introduction port extends to and is in fluidic communication with the periphery and the reagent zone comprises one or more reagents (i) mobilizable by a liquid sample introduced to the reagent zone and (ii) configured to bind to and/or enable the detection of a target indicative of the presence or amount of a pathogen present in such liquid sample introduced to the reagent zone. The microfluidic device may be an end fill device. In some embodiments, the microfluidic device is any of the microfluidic devices disclosed or claimed in the '325 Application, wherein the sample application port of such microfluidic device is configured as the sample introduction port that extends to and is in fluidic communication with the periphery, e.g., as an end fill device.

In embodiments, a method includes introducing a collection swab tip into a liquid buffer, which liquid buffer optionally includes a blocking agent such as a protein-based blocking agent such as bovine serum albumin, the collection swab tip comprising a liquid sample, e.g., a nasal, salivary, throat, mid turbinate, nasopharyngeal, or vaginal sample, collected from a mammal, e.g., a human being. The collection swab tip is removed from the buffer, with the removed collection swab tip holding a mixture of the liquid sample and buffer. A sample application port of a microfluidic device is contacted with the mixture of liquid sample and buffer held by the collection swab tip. The sample application zone may be disposed at a periphery of the microfluidic device, e.g., may be configured as an end fill microfluidic device. An amount of the mixture held by the collection swab tip flows from the swab tip through the sample application zone and into the microfluidic network of the microfluidic device, e.g., by capillary flow. The liquid that has flowed into the microfluid network is combined with one or more mobilizable reagents disposed within the microfluidic network. The presence or amount of one or more targets in the mixture is determined based upon an interaction within the microfluidic network between each of the one or more targets and at least one of the reagents, wherein each of the targets is indicative of the presence of a respective pathogen present in the liquid sample. The microfluidic device may be any of the microfluidic devices disclosed or claimed in the '325 Application, wherein the sample application port of such microfluidic device may be configured as a sample introduction port that extends to and is in fluidic communication with a periphery of the microfluidic device, e.g., a microfluidic device of the '325 Application configured as an end fill device.

In embodiments, a method includes introducing a collection swab tip into a liquid buffer, which liquid buffer optionally comprises a blocking agent such as a protein-based blocking agent such as bovine serum albumin. The collection swab tip includes a liquid sample, e.g., a nasal, salivary, throat, mid turbinate, nasopharyngeal, or vaginal sample, collected from a mammal, e.g., a human being. The buffer into which the collection swab tip is introduced has a total volume of about 225 microliters or less, 200 microliters or less, about 175 microliters or less, about 150 microliters or less, or about 125 microliters or less. The collection swab tip is removed from the buffer, the removed collection swab tip holding a mixture of the liquid sample and buffer. A sample application zone of a microfluidic device is contacted with the mixture held by the collection swab tip, thereby permitting an amount of the mixture to flow by capillary action from the swab tip through the sample application zone and into a microfluidic network of the microfluidic device. The sample application zone may be configured as peripheral sample application zone/port? in fluidic communication with a periphery of the microfluidic device. The presence or amount of one or more targets in the mixture is determined based upon an interaction within the microfluidic network of each of the one or more targets and at least one of the reagents, wherein each of the targets is indicative of the presence of a respective pathogen present in the liquid sample. The microfluidic device may be any of the microfluidic devices disclosed or claimed in the '325 Application, wherein the sample application port of such microfluidic device may be configured as a sample introduction port that extends to and is in fluidic communication with a periphery of the microfluidic device, e.g., a microfluidic device of the '325 Application configured as an end fill device.

In embodiments, a system includes an instrument including (i) a microfluidic device introduction port, (ii) a permanent magnet disposed in a fixed, e.g., operatively immovable, position with respect to the introduction port and (iii) an optical light source disposed in a fixed, e.g., operatively immovable (fixed), position within respect to the introduction port. A microfluidic device is received within the introduction port and disposed in an operatively secured state therein, the microfluidic device including (i) a microfluidic network disposed therein, (ii) a first reagent disposed within the microfluidic network, the first reagent comprising a first portion configured to bind a target, e.g., a biomolecule indicative of a pathogen, and a second portion comprising a magnetic particle, (iii) a second reagent disposed within the microfluidic network, the second reagent comprising a first portion configured to bind the target, e.g., forming an immunological sandwich with the first reagent, and a second portion comprising an optically detectable label, and (iii) a detection zone disposed within the microfluidic network. When the microfluidic device is disposed in the operatively secured state within the sample introduction port (i) the detection zone experiences a magnetic field emitted by the magnet, the magnetic field being sufficient to retain the first reagent, e.g., as a sandwich comprising the first reagent, the target, and the second reagent, within the detection zone if present in a liquid solution passing therethrough and (ii) the optical light source is configured to illuminate the detection zone.

In embodiments, a method includes providing a collection swab having a collection swab tip, the collection swab tip having been used to collect a biological specimen from a mammal, e.g., a human being. The biological specimen may include, for example, at least one of a nasal, salivary, throat, nasopharyngeal, mid turbinate, urine, or vaginal specimen. The collection swab tip with the collected biological specimen is introduced into a liquid, e.g., a buffer such as a viral transport medium. The liquid may include a blocking agent such as BSA. The collection swab tip is removed from the liquid buffer, with the collection swab tip holding a liquid mixture of the biological specimen and the liquid buffer. The collection swab tip removed from the liquid buffer is applied to a sample application port of a microfluidic device whereupon liquid mixture from the collection swab tip flows by capillary action through the sample application port and into a microfluidic network of the microfluidic device. Within the microfluidic network, the liquid mixture combines with at least one reagent. The at least one reagent may be configured to interact with, e.g., bind to, a target present in the biological specimen, such as a target indicative of the presence and/or amount of a pathogen. The at least one reagent may be, for example, any of the reagents disclosed in the '325 Application. The presence and/or amount of the pathogen in the liquid mixture is determined based upon the interaction between the at least one reagent and the target.

In embodiments, a method includes positioning a microfluidic device with respect to a magnet, wherein the microfluidic device includes a microfluidic network disposed therein. The magnet subjects only a portion of the microfluidic network to a magnetic field. The microfluidic network includes at least one mobilizable reagent including a magnetic particle disposed therein. The at least one reagent is configured to bind either directly or indirectly with a target, e.g., a target indicative of a pathogen. The microfluidic device may be, e.g., any of the microfluidic devices disclosed herein. The at least one mobilizable reagent may include, e.g., reagents configured to perform an immunological assay such as a competitive or sandwich immunoassay for the target. The reagents may be, e.g., any of the reagents disclosed in the '325 Application.

A liquid sample is introduced into the microfluidic network of the microfluidic device via a sample application port thereof. The liquid sample may be suspected of including the target and may be any of the liquid samples disclosed herein. The liquid sample may be introduced by any method disclosed herein, e.g., via the application of a porous member to the sample introduction port and/or capillary action within the sample application port and microfluidic network may draw the sample thereinto. At least some of the introduced liquid sample is combined with the at least one mobilizable reagent within the microfluidic network.

At least some of the liquid sample with the at least one mobilizable reagent is moved along the microfluidic network from a first portion thereof not subjected to the magnetic field and into the portion of the microfluidic network subjected to the magnetic field. In the first portion, the magnetic field is insufficiently strong to prevent the at least one reagent from being moved along with the liquid sample along the microfluidic network. Therefore, the at least one mobilizable reagent with magnetic particle may be moved along the microfluidic network without substantial retention of the reagent. In the portion of the microfluidic network subjected to the magnetic field, the magnetic field strength is high enough to substantially retain the at least one reagent with magnetic particle therein. As the liquid sample with magnetic particle reagent passes from the first portion not subjected to the magnetic field into the portion subjected to the magnetic field, the magnetic particle reagent is typically retained within an area that has a length along the longitudinal axis of the channel that is short compared to the full length of the channel. At least some of the remaining liquid sample, including any unretained constituents, is moved through and beyond the zone in which the magnetic particle reagent is retained. For example, the entire remaining liquid sample, including any unretained constituents, may be moved along the channel until a distal proximal? gas-liquid interface of the liquid sample passes through and beyond the zone in which the magnetic particle reagent is retained. Once the distal gas-liquid interface has passed through and beyond the zone, the retained magnetic particle reagent therein is exposed to the gas of the gas-liquid interface, e.g., exposed to the ambient air surrounding the microfluidic device.

The presence and/or amount of retained magnetic particle reagent is then determined, e.g., via an optical technique such as fluorescence or colorimetry. The determination may be performed using an optical detector such as a photodiode or by unassisted eye. The presence and/or amount of retained magnetic particle reagent is indicative of the presence and/or amount of the target in the liquid sample.

In the foregoing method, the steps of combining the introduced liquid sample with the at least one mobilizable reagent and moving the liquid sample with mobilizable reagent from the first portion of the microfluidic network to the portion of the microfluidic network subjected to the magnetic field are performed after the step of positioning and without substantially modifying either the position of the microfluidic device and magnet with respect to one another or substantially modifying the magnetic field experienced by the portion of the microfluidic network subjected to the magnetic field. In some embodiments, the step of determining the presence and/or amount of retained magnetic particle reagent is also performed after the step of positioning and without substantially modifying either the position of the microfluidic device and magnet with respect to one another or substantially modifying the magnetic field experienced by the portion of the microfluidic network subjected to the magnetic field. In some embodiments, the step of introducing the liquid sample to the microfluidic network is also performed after the step of positioning and without substantially modifying either the position of the microfluidic device and magnet with respect to one another or substantially modifying the magnetic field experienced by the portion of the microfluidic network subjected to the magnetic field.

Any of the collection swabs disclosed herein, e.g., as used in any of the methods disclosed herein, may have a tip that (i) includes a plurality of fibers, e.g., as a flocked swab tip or a spun fiber swab tip, (ii) includes a sponge or foam, (iii) is a sintered swab tip, (iv) is a three-dimensional printed swab tip, or (v) includes a combination of two or more swab tips of clauses (i) (iv).

Any of the porous members disclosed herein may comprise a porous network configured to retain, e.g., via surface tension and/or capillary forces, a liquid to be applied to a microfluidic device without inadvertent loss of liquid, e.g., by dripping from the porous member. The porous member is configured to permit liquid retained therein to be drawn by capillary action from the porous member into a microfluidic network of a microfluidic device. Exemplary materials are formed, e.g., as a porous membrane, flocked or spun fibers, a foam, a sponge, a 3D-printed network, a sintered medium or combination thereof. Exemplary materials include polymers such as urethanes, polyurethanes, polytetrafluoroethylene, polypropylene and combinates thereof.

In any of the embodiments disclosed herein, a target may be, e.g., a target indicative of the presence of a pathogen, such as a virus, bacterium, or fungus. Exemplary targets are indicative of a pathogen that associated with a respiratory condition such as an influenza virus, respiratory syncytial virus, or a coronavirus, e.g., SARS-CoV-2. Other exemplary targets include pathogens associated with gastrointestinal conditions such as norovirus.

1.-92. (canceled)

93. A method, comprising:
moving a liquid along a microchannel of a microchannel network of a microfluidic device by increasing or decreasing a pressure of a first gas disposed within the microchannel network and in gaseous communication with a first liquid-gas interface of the liquid, wherein the increasing or decreasing is performed by contacting a first location of an exterior surface of the microfluidic device with a first actuator, the first location overlying a volume of the first gas; and
independently of the increasing or decreasing the pressure of the first gas, oscillating a pressure of a second gas disposed within the microchannel network and in gaseous communication with a second liquid-gas interface of the liquid at a rate and amplitude sufficient to induce mixing of the liquid and a reagent in contact with the liquid within the microchannel, wherein the oscillating is performed by contacting a second location of an exterior surface of the microfluidic device with a second actuator, the second location overlying a volume of the second gas.

94. The method of claim 93, wherein the first and second locations are spaced apart from one another.

95. The method of claim 94, comprising performing one of the steps of (i) increasing or decreasing the pressure of the first gas and (ii) oscillating the pressure of the second gas, with the respective first or second actuator without simultaneously actuating the other of the first or second actuator.

96. The method of claim 94, wherein the moving the liquid comprises moving the first liquid-gas interface of the liquid along the microchannel over a distance of at least about 2 mm.

97. The method of claim 96, wherein the distance is about 12.5 mm or less.

98. The method of claim 96, wherein the moving the first liquid-gas interface over the distance comprises moving the first liquid-gas interface continuously over the distance.

99. The method of claim 94, wherein the moving the liquid comprises moving the first liquid-gas interface of the liquid along the microchannel over a distance of at least about 4 mm.

100. The method of claim 99, wherein the distance is about 12.5 mm or less.

101. The method of claim 94, wherein the first gas and the second gas are the same type of gas.

102. The method of claim 101, wherein the first gas and the second gas is air.

103. The method of claim 102, wherein the microfluidic device comprises a sample application zone in fluid communication with the microchannel and configured to receive the liquid, wherein the liquid within the microchannel and a third gas define a proximal gas-liquid interface disposed adjacent to or at the sample application zone, the third gas being air.

104. The method of claim 94, wherein the liquid comprises a biological specimen collected from a human being.

105. The method of claim 104, wherein the liquid comprises a mixture of a liquid buffer and the biological specimen.

106. The method of claim 105, wherein the biological specimen comprises, a nasal specimen, salivary specimen, throat specimen, nasopharyngeal specimen, mid turbinate specimen, urine specimen, vaginal specimen, or a combination thereof.
